(19) United States
(12) Patent Application Publication
(10) Pub. No.: US 2006/0252761 A1
(43) Pub. Date: Nov. 9, 2006

(54) Augmentation of extinction via administration of sub-antimicrobial doses of D-cycloserine

(76) Inventors: Michael Davis, Stone Mountain, GA (US); Kerry J. Ressler, Atlanta, GA (US); Jason P. McDevitt, Williamsburg, VA (US)

Correspondence Address: JASON P. MCDEVITT, 124 COUNTRY CLUB DRIVE, WILLIAMSBURG, VA, 23188, US

(21) Appl. No.: 11/347,937

(22) Filed: Feb. 6, 2006

Related U.S. Application Data
(63) Continuation-in-part of application No. 11/024,921, filed on Dec. 29, 2004.
(63) Continuation-in-part of application No. 10/924,591, filed on Aug. 24, 2004.
Application No. 10/924,591 is a continuation-in-part of application No. 10/473,640, filed on Apr. 22, 2004.
(63) Continuation-in-part of application No. PCT/US04/24841, filed on Aug. 3, 2004.
(60) Provisional application No. 60/651,114, filed on Feb. 8, 2005.
(60) Provisional application No. 60/667,140, filed on Mar. 31, 2005.
(60) Provisional application No. 60/533,003, filed on Dec. 29, 2003.
(60) Provisional application No. 60/625,253, filed on Nov. 5, 2004.
(60) Provisional application No. 60/363,991, filed on Mar. 13, 2002.
(60) Provisional application No. 60/279,868, filed on Mar. 29, 2001.
(60) Provisional application No. 60/492,795, filed on Aug. 6, 2003.

(51) Int. Cl. A61K 31/498; A61B 19/00

(52) U.S. Cl. 514/250; 128/898

(57) ABSTRACT

Methods are provided for facilitating psychological extinction of a deleterious, high-anxiety response that is disproportionate to the threat offered by a given stimulus. An afflicted subject is treated with a sub-antimicrobial dose of D-cycloserine in conjunction with extinction training. The methods are relevant for treatment of anxiety disorders, including phobic disorders and PTSD, in addition to other afflictions such as insomnia and erectile dysfunction.

### CROSS-REFERENCE TO RELATED APPLICATIONS

This application claims priority to U.S. Provisional Application No. 60/651,114, filed Feb. 8, 2005, and U.S. Provisional Application No. 60/667,140, filed Mar. 31, 2005; and additionally is a continuation-in-part of application Ser. No. 11/024,921, filed Dec. 29, 2004, which claims priority to U.S. Provisional Application No. 60/533,003, filed Dec. 29, 2003, and U.S. Provisional Application No. 60/625,253, filed Nov. 5, 2004; and additionally is a continuation-in-part of application Ser. No. 10/924,591, filed Aug. 24, 2004, which is a continuation-in-part of application Ser. No. 10/473,640, filed on Apr. 22, 2004, which is a national stage application under 35 U.S.C. 371 of International Application PCT/US02/09467, filed Mar. 28, 2002, which claims priority to U.S. Provisional Application No. 60/363,991, filed Mar. 13, 2002, and U.S. Provisional Application No. 60/279,868, filed Mar. 29, 2001; and additionally is a continuation-in-part under 35 U.S.C. 111(a) of International Application PCT/US2004/024841, filed Aug. 3, 2004, which claims priority to U.S. Provisional Application No. 60/492,795, filed Aug. 6, 2003.

### STATEMENT REGARDING FEDERALLY SPONSORED RESEARCH OR DEVELOPMENT

This invention was made with U.S. Government support under grant MH057250 awarded by the National Institutes of Health. The U.S. Government may have certain rights in this invention.

### BACKGROUND OF THE INVENTION

The invention relates to the treatment of medical disorders by facilitating, in a mammalian subject, psychological extinction of abnormally high-anxiety responses to stimuli.

Classical fear conditioning occurs when an affectively neutral stimulus is paired with a noxious aversive stimulus (unconditioned stimulus [US]) such as footshock. Afterward, the previously neutral stimulus (i.e., now the conditioned stimulus [CS]) is able to elicit a variety of autonomic, hormonal, and skeletal responses that accompany the conscious experience of fear in humans and which are used to operationally define fear in laboratory animals. The fear-eliciting properties of the CS can be extinguished by repeatedly presenting the CS in the absence of the US. It is generally believed that extinction does not reflect unlearning of the original association but involves instead the formation of new associations that compete with the previously conditioned response.

N-methyl-D-aspartate (NMDA) receptor antagonists have been shown to block extinction when administered either systemically or infused directly into the amygdala (as reviewed in Davis et al. (2005), Current Directions in Psychological Science, 14(4): 214-219), raising the question of whether NMDA agonists or partial agonists could facilitate extinction.

A reduced ability to extinguish high-anxiety responses resulting from fear memories is a significant clinical problem for a wide range of anxiety disorders including specific phobias, panic disorder, and post-traumatic stress disorder. These disorders are characterized by a high-anxiety response to a stimulus that is highly disproportionate to the threat. Treatment for these disorders often relies upon the progressive extinction of the high-anxiety response to the stimulus, and hence pharmacological enhancement of extinction could be of considerable clinical benefit in these conditions.

A reduced ability to extinguish deleterious, high-anxiety responses also contributes to recurring medical afflictions such as erectile dysfunction, insomnia, and chronic pain. While these afflictions have widely varying etiologies and symptoms, they share a common feature, which is that their severity and frequency of the afflictions can be exacerbated by anxiety regarding the affliction. For example, an episode of impotence in a male may generate significant anxiety about the condition, which may contribute to future episodes of impotence. Approved drugs for recurrent conditions such as insomnia and erectile dysfunction target the physiology of the symptoms, but neglect the mental component of the disorder.

### BRIEF SUMMARY OF THE INVENTION

Methods are provided for facilitating, in a mammalian subject, extinction of deleterious, high-anxiety responses to stimuli. The methods comprise administering sub-antimicrobial concentrations of D-cycloserine (DCS) to a subject in conjunction with extinction training. The extinction training is designed to develop a new, non-deleterious response to a given stimulus that previously generated disproportionate anxiety, i.e., to extinguish the high-anxiety response by replacing it with a more appropriate response. DCS facilitates extinction, and thus speeds up the process, thereby improving the therapeutic treatment. The methods are useful for treating a variety of afflictions for which extinction of deleterious anxiety responses would be beneficial, including anxiety disorders, sexual dysfunction, chronic pain, and insomnia.

### BRIEF DESCRIPTION OF THE SEVERAL VIEWS OF THE DRAWINGS

FIG. 1 shows the parametric evaluation of different amounts of extinction training. A. Timeline of the behavioral procedures for Experiment 1. B. Percent fear-potentiated startle measured 24 hours before (pre-test) and 24 hours after (post-test) extinction training or context exposure. The control group was tested 2 days after the pre-test, with no intervening exposures. One session of non-reinforced cue exposure produced only modest levels of extinction. Two or three sessions more completely extinguished the fear response. *p<0.05 versus context exposure group; p<0.05 versus control group.

FIG. 2 shows the dose-response function for the effect of DCS on extinction. A. Timeline of the behavioral procedures for Experiment 2. B. Percent fear-potentiated startle measured 24 hours before and 24 hours after a single session of extinction training in rats injected with saline or DCS (3.25, 15, or 30 mg/kg, i.p.) 30 minutes prior to non-reinforced cue exposure. DCS dose-dependently facilitated extinction learning. * p<0.05 versus saline post-extinction.

FIG. 3 shows the effect of DCS in non-extinguished rats. A. Timeline of the behavioral procedures for Experiment 3. B. Percent fear-potentiated startle measured 24 hours before and 24 hours after extinction training. Saline or DCS (15 mg/kg, i.p.) was administered 30 minutes prior to a single session of either extinction training (cue exposure) or context alone exposure. Fear-potentiated startle was significantly lower in rats that received DCS+extinction training than in rats that received saline+extinction training. Fear-potentiated startle was not appreciably affected by DCS in rats that did not receive extinction training. *p<0.05 versus saline+extinction training.

FIG. 4 shows the effect of the strychnine-insensitive glycine recognition site antagonist HA-966 on extinction and on the facilitation of extinction by DCS. A. Timeline of the behavioral procedures for Experiment 4. B. Percent fear-potentiated startle measured 24 hours before (pre-extinction test) and 24 hours after (post-extinction test) extinction training. Saline or HA-966 (6 mg/kg, i.p.) were administered 10 minutes before a second injection of saline or DCS, followed 30 minutes later by a single session of extinction training. HA-966 completely blocked the effects of DCS but did not, on its own, noticeably influence extinction at this dose. *p<0.05 versus all other groups.

FIG. 5 shows the effect of pre-test DCS and HA-966 administration on fear-potentiated startle. A. Timeline of the behavioral procedures for Experiment 5. B. Percent fear-potentiated startle measured 24 hours after fear-conditioning in rats receiving pre-test injections of saline, DCS (15 mg/kg), or HA-966 (6 mg/kg). Neither drug had any discernible effect on fear-potentiated startle.

FIG. 6 shows cannula tip placements transcribed onto atlas plates adapted from Paxinos and Watson ((1997) The Rat Brain in Stereotaxic Coordinates (3^{rd} ed., Academic Press, New York)). The distance from bregma is indicated to the left; nuclei within the plane of section are identified to the right. BM=basomedial amygdaloid nucleus; BL=basolateral amygdaloid nucleus; BLV=basolateral amygdaloid nucleus, ventral part; CeM=central amygdaloid nucleus, medial division; CeL=central amygdaloid nucleus, lateral division; ic=internal capsule; LA=lateral amygdaloid nucleus; OPT=optic tract.

FIG. 7 shows the effect of intra-amygdala DCS infusions. A. Timeline of the behavioral procedures for Experiment 3. B. PBS or DCS (10 μg/side) was infused into the amygdala 15 minutes prior to extinction training. Other rats received DCS without extinction training. When tested 24 hours later, fear-potentiated startle was significantly lower in rats that received DCS+extinction training than in rats that received PBS+extinction training. Fear-potentiated startle was not appreciably affected by DCS in rats that did not receive extinction training. For the group that received DCS without extinction training, mean percent potentiation was calculated with and without data from a single outlier who had an atypically high percent potentiation score. *p<0.05 versus all other groups.

FIG. 8 is a composite figure showing absolute startle values for all rats receiving drugs prior to extinction training. Shaded bars indicate baseline startle amplitude on noise alone trials; open bars indicate startle amplitude on light-noise trials. The difference between these two (i.e., fear-potentiated startle) is indicated by the striped bars. In no case were significant differences found in baseline startle during the fear-potentiated startle test 24 hours after drug administration. Moreover the statistical results were similar when absolute difference scores (i.e., startle amplitude on light-noise trials minus startle amplitude on noise alone trials) rather than percent potentiation scores were analyzed. *p<0.05 (except Panel C, p=0.087) versus all left-most bars.

FIG. 9 shows measures of acrophobia within the virtual environment. A. Level of fear as measured by subjective units of discomfort (SUDS 1=no fear, 100=maximum fear) during the pre-treatment assessment at each successive floor in the virtual glass elevator. B. SUDS during the first treatment session in which subjects elevated to successive floors at 5-minute intervals. C. Floor to which the subjects elevated at 5-minute intervals during the first treatment session. There were no significant differences between the groups during the pretreatment SUDS measure or either measure during the first treatment session.

FIG. 10 shows improvement of acrophobia within the virtual environment with DCS. A. Reduction in fear from pre to post-test following the two therapy sessions measured at the first follow-up assessment. Decrease in SUDS level (y-axis) is shown for each floor (1-19) of the virtual glass elevator. Overall ANOVA was performed using pre-post difference and floor as within-subjects variables and drug group as between-subjects variable. Significant overall pre-post changes were seen: F(1,25)=38, p≦0.001. Significant effect of floor was found: F(6, 150)=89, p≦0.001. Most importantly, significant effect of pre-post X floor X drug interaction was found: F(6,150)=3.8, p≦0.001. B. Change in SUDS from pre to post-test at the 3-month long-term follow-up assessment. Statistics were performed as above. Significant overall pre-post changes were seen: F(1,17)=21, p≦0.001. Significant effect of floor was found: F(6, 102)=81, p≦0.001. Most importantly, significant effect of pre-post X floor X drug interaction was found: F(6,102)=2.4, p≦0.05.

FIG. 11 shows physiological measures of anxiety within the virtual environment. Spontaneous fluctuations in baseline skin conductance levels are shown as a function of acrophobia treatment response and treatment condition. A. Subjective improvement in acrophobia symptoms. Those reporting improvement in symptoms show significantly lower post-treatment spontaneous fluctuations in the virtual environment (F(1,19)=4.5, p≦0.05). B. Decreased avoidance (self-reports of whether they have self-exposed to heights since treatment) also was associated with significantly lower spontaneous fluctuations of skin conductance (F(1,19)=8.26; p≦0.01). C. Subjects treated with DCS during exposure therapy showed significant decreases in post-treatment fluctuations (paired t-test, p≦0.05) compared to those treated with placebo (p≧0.5).

FIG. 12 shows reduction in fear compared to pretreatment baseline on general measures of acrophobia in the real world, 1 week after the first therapy session (mid-treatment), 1-2 weeks after the second therapy session (Post 1 week), or at 3-month follow-up (Post 3 month). A. Acrophobia Avoidance Questionnaire, (repeated measures ANOVA, DCS vs Placebo: F(1,19)=6.1, p≦0.02). B. Acrophobia Anxiety Questionnaire, (repeated measures ANOVA, DCS vs Placebo: F(1,19)=7.9, p≦0.01). C. Attitude Towards Heights Inventory, (repeated measures ANOVA, DCS vs Placebo: F(1,19)=4.9, p≦0.04).

FIG. 13 shows measures of global improvement and self exposure. A. Average Clinical Global Improvement scores (CGI, 1=“Very much improved”, 4=“No Change”) for placebo vs. DCS groups at 1 week and 3 months following treatment. (repeated measures ANOVA, DCS vs Placebo: F(1,19)=11.6, P≦0.01). B. Percentage of subjects rating themselves as “Very Much Improved” or “Much Improved” on the CGI. Subjects receiving DCS during treatment demonstrated significantly greater subjective improvement compared to those receiving placebo. (repeated measures ANOVA, DCS vs placebo demonstrating an overall drug effect but no drug×time interaction; F(1,19)=11.5, p≦0.01). C. Reduction in acrophobia as measured by real-world self-exposures to heights during the 3 months following treatment. Subjects receiving DCS during treatment demonstrated significantly more exposures to heights at 3 months than did subjects receiving placebo (F(1,18)=7.7, p≦0.01).

### DETAILED DESCRIPTION OF THE INVENTION

The present invention is directed to methods for facilitating extinction of a deleterious, high-anxiety response to a psychological stimulus. The methods comprise administering to a subject a sub-antimicrobial amount of DCS in conjunction with extinction training.

As used herein, each of the following terms has the meaning associated with it as described below.

The articles “a” and “an” are used herein to refer to one or to more than one (i.e. to at least one) of the grammatical object of the article. By way of example, “an element” means one element or more than one element.

As used herein, “plurality” means at least two.

As used herein, “FDA” means the United States Food and Drug Administration.

Any ranges cited herein are inclusive, e.g., “between about 50 mg and 100 mg” includes compositions of 50 mg and 100 mg.

As used herein, “acute” administration of DCS means a single exposure within an extended time period of the subject to the therapeutically effective amount of DCS that facilitates extinction. In conjunction with this definition of “acute”, an extended time period is defined as four days or longer, e.g., once-weekly administration of DCS constitutes acute administration. Administering a dose of DCS to a subject, followed by a second dose 24 hours later, does not constitute acute dosing. Administering a single dose of DCS, wherein the dose is formulated to have both immediate release and delayed release characteristics, constitutes acute dosing provided that the peak blood level of DCS in the subject is achieved within 12 hours of the time the dose is administered.

As used herein, a subject is “treated”, or subjected to “treatment”, when an earnest attempt is made to alleviate a medical disorder or disease. For example, a subject can be treated for a disorder by being administered a pharmacologic agent that is intended to alleviate the disorder, irrespective of whether the treatment actually was successful in alleviating the disorder.

As used herein, a disease or disorder or medical affliction is “alleviated” if either (or both) the severity or frequency of a symptom of the disease or disorder or medical affliction is reduced.

A “subject” of diagnosis or treatment is a mammal.

A “therapeutic” treatment is a treatment administered to a subject who exhibits signs of pathology for the purpose of diminishing or eliminating those signs.

A “therapeutically effective amount” or “therapeutically effective dose” of the pharmacologic agent is an amount of the pharmacologic agent that, when administered in conjunction with extinction training, results in an improved therapeutic benefit relative to that observed with extinction training in the absence of administering the pharmacologic agent.

As used herein, a “deleterious, high-anxiety response” refers to a subject's response to a given stimulus, wherein the response is characterized by a high level of anxiety that is disproportionate to the threat represented by the stimulus. Accordingly, a stimulus that generates little if any anxiety in most subjects would generate substantial anxiety in a subject undergoing a deleterious, high-anxiety response. These deleterious, high-anxiety responses cause or exacerbate symptoms characteristic of the medical disorders described herein.

As used herein, the term “neuropathic pain” means pain that originates from a damaged or malfunctioning nerve or nervous system. “Chronic pain” means pain that has lasted for more than three months, generally resulting in significant psychological and emotional affects and limiting a person's ability to fully function.

The term “excessive daytime sleepiness” is a condition in which an individual feels very drowsy during the day and has an overwhelming urge to fall asleep, even after getting enough nighttime sleep. People with excessive daytime sleepiness frequently doze, nap, or fall asleep in situations where they need or want to be fully awake and alert. This can be particularly dangerous at times, such as when driving a car or operating other hazardous machinery. Excessive daytime sleepiness can interfere significantly with an individual's ability to concentrate and perform daily tasks and routines. People with excessive daytime sleepiness often report feelings of low self-esteem, frustration, and anger about being misunderstood and regarded as unintelligent, lazy or uninterested in learning. Excessive daytime sleepiness is a sign of an underlying medical condition, typically, although not necessarily, a sleep disorder such as narcolepsy, sleep apnea, circadian rhythm disorder, nighttime insomnia, and restless legs syndrome. The term “narcolepsy” is traditionally defined as a sleep disorder characterized by excessive sleepiness, cataplexy, sleep paralysis, hypnogogic hallucinations, and an abnormal tendency to pass directly from wakefulness into REM sleep. For the purposes of this patent application, the term “excessive daytime sleepiness” is inclusive of the term “narcolepsy”, and therefore methods and compositions useful for treating excessive daytime sleepiness are also useful for treating narcolepsy.

As used herein, “attention deficit-hyperactivity disorder”, sometimes referred to as attention deficit disorder, is defined as a disorder in individuals characterized by serious and persistent difficulties relating to inattentiveness, distractability, impulsivity, and hyperactivity.

As used herein, “chronic fatigue syndrome” refers to a disorder characterized by extreme exhaustion, muscle pain, cognitive problems and a number of other physical symptoms.

As used herein, “insomnia” is defined as the inability to fall asleep or to stay asleep for a sufficient amount of time during regular sleeping hours. It includes acute insomnia, which occurs in either a transient or short term form, and chronic insomnia. It also includes initial insomnia, defined as difficulty in falling asleep; middle insomnia, defined as awakening in the middle of the night followed by eventually falling back to sleep, but with difficulty; and terminal insomnia, defined as awakening before one's usual waking time and being unable to return to sleep.

As used herein, “biofeedback” refers to a technique in which subjects are trained to improve their health by using signals from their own bodies to control their own physiological responses. Biofeedback is particularly useful in enabling subjects to learn to control physiological processes that normally occur involuntarily, such as blood pressure, heart rate, muscle tension, and skin temperature.

As used herein, “erectile dysfunction” is impotence resulting from a man's inability to obtain or maintain an erection of his penis.

As used herein, “sexual performance” includes measures widely ascribed to sexual performance. For example, in men, improved sexual performance can entail:
(1) reduction or elimination of premature ejaculation;
(2) improved sexual stamina; and/or
(3) enhanced ability to achieve multiple erections;
   in addition to other criteria known in the art.

As used herein, “female sexual dysfunction” is characterized by an unwanted lack of desire, arousal, or orgasm. The American Psychological Association (APA) classifies female sexual problems as mental disorders: loss of sexual desire or arousal, discomfort during intercourse, diminished blood flow to the vagina, trauma-related aversion to sex, and the inability to achieve orgasm.

As used herein, the term “NMDA receptor” refers to the N-methyl-D-aspartate glutamate receptor subtype and the “NMDA channel” refers to the ion channel that may open when glutamate binds to the NMDA receptor (Yamakura and Shimoji (1999) Prog. Neurobiol. 59(3):279-298).

The term “agonist” generally refers to a compound that interacts with a receptor and initiates or facilitates a response characteristic of what happens when the endogenous ligand binds to that receptor. The term “antagonist” generally refers to a compound that interacts with a receptor and initiates or facilitates a response counter to what happens when the endogenous ligand or an agonist binds to that receptor.

As used herein, “anxiety disorder” refers to a disorder characterized by fear, anxiety, addiction, and the like that can be treated with the methods of the invention. An individual who can benefit from the methods of the invention may have a single disorder, or may have a constellation of disorders. The anxiety disorders contemplated in the present invention include, but are not limited to, fear and anxiety disorders, addictive disorders including substance-abuse disorders, and mood disorders. Fear and anxiety disorders include, but are not limited to, panic disorder, agoraphobia, social phobia, specific phobia, post-traumatic stress disorder (PTSD), obsessive-compulsive disorder, and movement disorders such as Tourette's syndrome. The disorders contemplated herein are defined in, for example, the DSM-IV (Diagnostic and Statistical Manual of Mental Disorders (4th ed., American Psychiatric Association, Washington D.C., 1994)).

As used herein, a “B-complex vitamin” is one or more vitamins selected from the group comprising thiamine (B1), riboflavin (B2), niacin (B3), pyridoxine (B6), folic acid (B9), cyanocobalamin (B12), pantothenic acid and biotin.

“Pharmacologic agent” refers to a compound, mixture, etc., exhibiting properties indicating usefulness in a medicament.

As used herein, two pharmacologic agents are said to be “co-administered” when the pharmacologic agents are administered simultaneously in a single dosage form, or administered separately within a limited period of time of about three hours.

“Extinction training” refers to a method wherein a subject having deleterious, high-anxiety responses to a given stimulus, is exposed to the stimulus such that the conditions of the exposure are manipulated to control the outcome or otherwise reduce the likelihood of an event occurring that would tend to reinforce the fear response. The goal of extinction training is to pair the previously aversive stimulus with a new learning resulting from a non-deleterious outcome resulting from the stimulus, thereby generating, in future exposures to the stimulus, a more appropriate response in place of the previous deleterious, high-anxiety response. For example, the conditions of the exposure can be manipulated by psychotherapy or pharmacotherapy. In one example of extinction training, a subject having a phobic disorder undergoes extinction training by participating in a traditional exposure-based psychotherapy session. As another example, a subject having erectile dysfunction undergoes extinction training by taking a drug that treats the symptoms of erectile dysfunction (e.g., sildenafil) prior to engaging in a sexual interlude. As used herein, an “extinction learning event” refers to a completed stimulus/response extinction training cycle. For example, a dog conditioned to salivate to the sound of a bell (by pairing the bell with food) would experience an extinction learning event when the bell is sounded and the food is not presented. A subject could experience multiple extinction learning events within a given extinction training session. For example, an hour-long psychotherapy session could entail numerous extinction learning events.

“Psychotherapy” refers to a treatment of mental illness, anxiety disorders or emotional disturbances primarily by verbal or non-verbal communication.

The term “D-cycloserine”, or “DCS”, refers to the chemical D-cycloserine (CA Index Name: 3-Isoxazolidinone, 4-amino-, (4R)-(9CI); CAS Registry No. 68-41-7), or pharmaceutically acceptable salts thereof. DCS is an FDA-approved drug for treatment of tuberculosis, and is sold by Eli Lilly and Company under the trade name Seromycin®. DCS is a structural analog of D-alanine, and is a broad-spectrum antibiotic produced by some strains of Streptomyces orchidaceus and S. garphalus. DCS has antibiotic activity in vitro against growth phase Gram-negative bacteria such as Escherichia coli, some strains of Staphylococcus aureus, and Chlamydia species, among others. The minimum inhibitory concentrations (MIC) in vitro for typical Mycobacterium tuberculosis strains range from about 6-25 μg/mL.

DCS has been FDA-approved for over 20 years for the treatment of tuberculosis. It has been tested as a cognitive enhancer in several clinical trials over the last decade. For tuberculosis, DCS is generally dosed at 500-1000 mg/day divided twice daily (PDR 1997) with chronic treatment. At a dose of 500 mg/day, serum concentrations of 25-30 μg/ml are generally maintained. The peak serum concentrations occur within 3-8 hours after dosing, and it is primarily renally excreted with a half-life of 10 hours.

At doses typically used to treat tuberculosis, DCS can give rise to significant neurological side effects in treated subjects. Typical side effects on chronic dosing schedules (who were generally chronically ill with tuberculosis) include drowsiness, depression, headache, confusion, tremor, vertigo, and memory difficulties, paresthesias, and seizure. Per the methods of the inventions, these side effects will be greatly reduced by infrequent dosing at concentrations five to twentyfold lower than those routinely used for TB treatment. For example, in a recent pilot clinical study looking at use of different daily dosages on DCS for treatment of autism (Am J Psychiatry (2004), 161:2115-2117), DCS was generally well-tolerated and resulted in significant improvement in social withdrawal. However, two patients receiving high-dose DCS (2.8 or more mg/kg/day) experienced adverse effects consisting of a motor tic and increased echolalia. A long history of clinical studies with DCS have, not surprisingly, demonstrated a correlation between increased dosages of DCS and increased side effects.

A “sub-antimicrobial” dose refers to a dose of DCS that is less than or equal to 2 mg DCS per kg body weight of the subject (i.e., less than or equal to 2 mg/kg), and greater than about 0.4 mg/kg (this lower limit is needed to maintain efficacy for facilitation of extinction). When administered to a subject, sub-antimicrobial doses of DCS achieve peak serum concentrations in the subject of less than or equal to about 5 μg/mL, although there is substantial variability between subjects. At these low concentrations of DCS, the drug no longer kills most microorganisms, including those that are ordinarily susceptible to higher DCS concentrations typically reached in the body when DCS is used to treat tuberculosis (i.e., 500 mg or 1000 mg per day). Obviously, the serum concentration levels of DCS in subjects administered DCS are a function of numerous factors, including body weight, metabolism, and the amount of drug ingested. Furthermore, all microorganisms do not have the same susceptibility to DCS. Accordingly, while it is possible that a sub-antimicrobial dose of DCS can still kill a small subset of microorganisms, sub-antimicrobial doses of DCS generally will not have a significant antimicrobial effect in the body. When administered to adult human subjects, a sub-antimicrobial dose of DCS generally comprises a drug formulation (e.g., pill, capsule, tablet) of DCS containing DCS in an amount equal to or less than 100 mg, preferably less than 80 mg DCS to provide a greater margin between the concentration of DCS and the minimum inhibitory concentration (MIC) of DCS against microorganisms active in a subject's body. Children being administered DCS for treatment of tuberculosis are normally dosed at a level between 10-20 mg/kg. A sub-antimicrobial dose of DCS, when administered to a child subject according to the methods of the present invention, comprises less than or equal to 2 mg/kg, and generally achieves peak serum concentrations of DCS in the child subject of less than or equal to 5 μg/mL.

In the United States, DCS is available in 250 mg capsules (two to four capsules per day are typically administered for treatment of tuberculosis). DCS is only approved by the FDA for use as an antibiotic. The present invention is directed to methods for facilitating extinction of a deleterious, high-anxiety response to a psychological stimulus. The methods comprise administering to a subject a sub-antimicrobial amount of DCS in conjunction with extinction training. Although bacteriocidal doses of DCS (i.e., dosages of greater than about 4 mg/kg)) may be effective in facilitating extinction of deleterious, high-anxiety responses to a stimulus in a given subject, dosing bacteriocidal concentrations of DCS is significantly disadvantaged relative to the methods of the present invention, which require sub-antimicrobial doses of DCS. Sub-antimicrobial doses of DCS are effective in facilitating extinction of said deleterious, high-anxiety response, and additionally:
1. give rise to reduced side effects relative to DCS doses exceeding 2 mg/kg; and
2. are not antimicrobial in the body, and therefore may be less likely to contribute to evolution of antibiotic resistance.

In short, administering to subjects a sub-antimicrobial dose of DCS to facilitate extinction is substantially safer than administering a higher DCS dose. Importantly, there is not a corresponding substantial decrease in efficacy, as is demonstrated in human clinical trials described later in the specification.

Administering Sub-Antimicrobial DCS

The timing of administration and the therapeutically effective dose of DCS in a given subject will depend on the severity of symptoms, in addition to the age, sex, and size of the subject being treated, among other variables. One of the most significant variables involves whether or not a subject remembers to take the drug. Ideally, a therapeutically effective level of the pharmacologic agent is present in the subject being treated at the commencement of extinction training.

At present, it has not been conclusively determined whether DCS works exclusively by consolidating extinction learning, or whether it additionally facilitates acquisition of learning. Nevertheless, it is clear that it is important for DCS to be absorbed in the subject at therapeutically effective levels at the time of an extinction learning event. Increasing the delay of administration of DCS after extinction training leads to a linear decrease in the ability of DCS to facilitate extinction (Ledgerwood et al., (2003), Behavioral Neuroscience (2003), Vol. 117(2): 341-349). In other words, the ability of DCS to consolidate extinction learning falls off directly with the time administered after an extinction learning event. Ideally, DCS is administered such that concentrations in the subject are at therapeutically effective levels at the time of an extinction learning event. Ideally, the serum concentration of DCS in a subject is at least 1 μg/mL at the time of an extinction learning event. In order for orally administered DCS to reach such therapeutically effective levels in the body of a subject at the time of completion of an extinction learning event, it is necessary to administer DCS prior to said extinction learning event, ideally at least 30 minutes prior to completion of said extinction learning event.

Consequently, administering a sub-antimicrobial dose of DCS to a subject undergoing extinction training via psychotherapy at the end of a psychotherapy session will be less effective for facilitating extinction than would be the case if the subject was administered DCS at the start of the session, or even in the middle of the session. That said, while it is preferable to administer DCS prior to extinction training, administering DCS to a subject after extinction training will still provide some benefits (albeit reduced), and such methods are contemplated in the invention. In particular, a subject may forget to take the DCS medication in advance of extinction training, in which case administering DCS after extinction training would be more beneficial than not administering DCS at all.

In general, the timing of administration of DCS according to the present invention will be within about eight hours, more preferably within about four hours, prior to an extinction learning event. The pharmacologic agent can also be administered within about four hours, more preferably within about 30 minutes following an extinction learning event. Accordingly, when DCS is administered to a subject “in conjunction with” extinction training, DCS is administered within eight hours prior to an extinction learning event, more preferably within about four hours prior to an extinction learning event, or somewhat less preferably, within about four hours after an extinction learning event. Accordingly, if a therapeutically effective, sub-antimicrobial dose of DCS is administered to a subject two hours prior to an extinction learning event, then the DCS will be present at therapeutically effective levels both before and after the extinction learning event.

Extinction Training

The goal of extinction training is to pair a stimulus that previously provoked a deleterious, high-anxiety response with a new learning that the stimulus will not lead to a negative outcome, thereby generating in the subject a new, more appropriate response to the stimulus to replace the previous disproportionate response.

Psychotherapy

The methods of the invention contemplate treatment of anxiety disorders by combining (i) administration of sub-antimicrobial DCS to a subject; and (ii) extinction training provided by any type of psychotherapy that is suitable for the particular medical affliction for which the subject is undergoing treatment. Suitable methods of psychotherapy include exposure-based psychotherapy, cognitive psychotherapy, and psychodynamically oriented psychotherapy.

One method of psychotherapy specifically contemplated is the use of virtual reality (VR) exposure therapy to treat an anxiety disorder using the methods of the invention. VR exposure therapy has been used to treat a variety of disorders including anxiety disorders such as the fear of heights (Rothbaum and Hodges (1999) Behav. Modif. 23(4):507-25), as well as specific phobias, eating disorders, and PTSD (Anderson et al. (2001) Bull. Menninger Clin. 65(1):78-91). Because of the prevalence of PTSD in the general population and the successful use of VR therapy to treat PTSD in, for example, Vietnam veterans (Rothbaum et al. (1999) J. Trauma Stress 12(2):263-71) or rape victims (Rothbaum et al. (2001) J. Trauma Stress 14(2):283-93), one embodiment of the present invention specifically contemplates the use of such VR exposure psychotherapy, in conjunction with sub-antimicrobial DCS, to facilitate extinction of deleterious, high-anxiety responses to neutral stimuli that are associated with PTSD.

Another method of psychotherapy that is particularly beneficial when utilized in accordance with the methods and compositions of the present invention is cognitive behavioral therapy (“CBT”). CBT is a form of psychotherapy that combines cognitive therapy and behavior therapy, and emphasizes the critical role of thinking in causing people to act and feel as they do. Therefore, if an individual is experiencing unwanted feelings and behaviors, CBT teaches that it is important to identify the thinking that is causing the undesirable feelings and/or behaviors and to learn how to replace this deleterious thinking with thoughts that lead to more desirable reactions. There are many approaches to cognitive-behavioral therapy, including Rational Emotive Behavior Therapy, Rational Behavior Therapy, Rational Living Therapy, Cognitive Therapy, and Dialectic Behavior Therapy. CBT generally begins with a review of a subject's past experiences with similar or different problems, leading to an understanding of the habitual and problematic manner of thinking and behaving that underlies the subject's problem, and culminating in a strategy to develop new ways of thinking, behaving and interacting to manage, alleviate, or eliminate the problem. CBT sessions involving a therapist and a subject typically take between 30 minutes and an hour, and are structured and directive. Typically, each session has a specific agenda. CBT can be used successfully to treat anxiety disorders (e.g., PTSD, agoraphobia, specific phobia, social phobia, substance abuse/addiction, and obsessive-compulsive disorder), depression, chronic pain, insomnia, sexual dysfunction, obesity, and eating disorders.

In one embodiment of the invention, subjects suffering from chronic pain undergo weekly cognitive behavioral therapy sessions to help alleviate the pain. At the outset of each session, subjects are administered DCS (75 mg). Relative to subjects treated only via cognitive behavioral therapy, chronic pain is expected to be reduced to a greater extent in subjects treated with a combination of cognitive behavioral therapy and acute doses of DCS. In a variation on this embodiment, the subjects are administered DCS after cognitive behavioral therapy. In another variation, the subjects are administered DCS after cognitive behavioral therapy only if the cognitive behavioral therapy yielded positive results on that day, as determined by the subject and/or therapist.

Biofeedback

Biofeedback is often aimed at changing habitual reactions to stress that can cause pain or disease. Biofeedback is particularly useful in enabling subjects to learn to control physiological processes that normally occur involuntarily, such as blood pressure, heart rate, muscle tension, and skin temperature.

Many clinicians believe that some of their patients have essentially forgotten how to relax. Feedback of physical responses such as skin temperature and muscle tension provides information that aids subjects in recognizing a relaxed state. For example, one commonly used biofeedback machine detects electrical signals in muscles, and translates these signals into a form that subjects can detect (e.g., flashing bulb, beeper). Subjects can learn to relax tense muscles by learning and repeating behaviors that generate the desirable response from the machine (e.g., reduced beeping, indicative of enhanced relaxation).

The three most common forms of biofeedback therapy are (1) electromyography (EMG), which measures muscle tension, (2) thermal biofeedback, which measures skin temperature, and (3) electroencephalography (EEG, neurofeedback), which measures brain wave activity.

Biofeedback has been demonstrated to be useful, or suggested to be useful, for a range of medical disorders including but not limited to: anorexia nervosa and other eating disorders, anxiety and depression, asthma, autism, back pain, chronic pain, bed-wetting, incontinence, fecal incontinence, constipation, diabetes, sexual disorders, Raynaud's disease, and ADHD.

By administering sub-antimicrobial DCS in conjunction with extinction training, the benefits of the biofeedback therapy can be enhanced. DCS will enhance consolidation of the response that is learned in biofeedback, thereby reducing the number of biofeedback sessions required to reach the same clinical endpoint and level of benefit to the subject. For example, the methods of the invention could be used as described in the following protocols.

In one embodiment of the invention, a subject suffering from chronic pain undergoes biofeedback sessions to help alleviate the pain. Upon conclusion of each session wherein the subject has made progress in learning/developing responses that reduce the chronic pain, the subject is administered DCS (75 mg) in order to consolidate the desired learning.

In another embodiment, a subject suffering from phantom limb syndrome undergoes thermal biofeedback sessions to reduce and hopefully eliminate the symptoms. At the start of each session, the subject is administered DCS (75 mg). The acute DCS treatment can provide benefits to the subject beyond those that would be obtained if the subject underwent biofeedback without supplemental DCS.

In another embodiment, a subject suffering from migraine headaches undergoes biofeedback sessions in order to increase physiological responses that decrease stress. In some sessions, thermal biofeedback is used to assist the subject in developing better control of the warming of her hands, a technique that has been found to be effective in controlling migraine symptoms. Upon conclusion of each session wherein the subject has made progress in learning/developing responses that improve control of hand warming, the subject is administered DCS (75 mg). The acute DCS treatment may provide benefits to the subject beyond those that would be obtained if the subject underwent biofeedback without supplemental DCS.

Extinction training does not require intervention of a trained specialist. Individuals can carry out extinction training on themselves.

Representative Afflictions

Anxiety Disorders

Anxiety disorders that may be treated with the methods and compositions of the present invention include, but are not limited to, fear and anxiety disorders, addictive disorders including substance-abuse disorders, and mood disorders. Fear and anxiety disorders include, but are not limited to, panic disorder, agoraphobia, social phobia, specific phobia, post-traumatic stress disorder (PTSD), obsessive-compulsive disorder, and movement disorders such as Tourette's syndrome.

According to the methods of the invention, subjects afflicted with anxiety disorders are treated by being administered sub-antimicrobial doses of DCS in conjunction with an extinction learning event. In preferred embodiments of the invention, the extinction learning event is achieved via psychotherapy. In such cases, the DCS is administered within eight hours of an extinction learning event, preferably within about four hours of an extinction learning event, more preferably within about four hours prior to an extinction learning event. In other embodiments, the extinction training is not obtained via psychotherapy. For example, a subject taking a drug for treatment of social phobia (e.g., a selective serotonin reuptake inhibitor) might occasionally have an extinction learning event characterized by uncommonly low anxiety with respect to said condition. The subject could then be administered sub-antimicrobial DCS to consolidate said learning. For a subject recovering from alcohol abuse, an extinction learning event could be exposure to a social setting typically associated with alcohol (e.g., a party or bar), wherein the subject did not have substantial anxiety with respect to a desire to drink alcoholic beverages.

For example, in one embodiment of the invention, a moderately introverted subject typically experiences anxiety when put in a situation that places a premium on social interactions with people whom she does not know well, for example, a cocktail reception at a business convention. The subject would like to be more relaxed in such situations, believing it would improve her job performance by improving her ability to network. The subject has noted that on previous occasions, her anxiety is reduced, and gregariousness increased, after she has one or more alcoholic drinks. Accordingly, she attends three typical social business functions over a one-month span. Each time, she consumes one alcoholic beverage, and makes an attempt to be as socially engaging as possible. She takes a DCS tablet (50 mg) within about four hours of the social business function, preferably prior to the start of said function. At subsequent social business functions, she is likely to experience reduced anxiety, and increased social confidence. In a variation on this embodiment, the subject could take a DCS tablet after, rather than before, the commencement of said function, and could take the DCS tablet only if the social encounter went well, as determined by the subject.

Panic disorder has been shown to be responsive to pharmaceutical treatment with SSRIs. Many subjects whose symptoms are alleviated by SSRIs would like to go off their medications without experiencing a revival of their symptoms. In one embodiment of the invention, a subject who responds positively to treatment with fluoxetine is also treated with DCS on an intermittent basis. Accordingly, a subject suffering from panic disorder is treated on a daily basis for 30 days by being administered two pharmacologic agents:
1. a coated tablet containing either DCS (100 mg) or placebo; and
2. fluoxetine, 20 mg.
   The first pharmacologic agent comes in a serially numbered pharmaceutical kit, with DCS tablets corresponding to days 5, 15, and 25, and placebo tablets on all other days. The placebo tablets and DCS tablets are visually indistinguishable. Relative to subjects treated with fluoxetine alone, a responsive subject receiving the combination therapy will, on average, have reduced anxiety, and may be less likely to experience a recurrence of the condition sufficient to require additional drug therapy.

In other embodiments, subjects with addictive disorders can be treated according to the methods of the invention. In one such example, subjects undergoing therapy for alcoholism are administered 50 mg DCS on an acute basis in conjunction with therapy.

In preferred embodiments, sub-antimicrobial DCS is administered on an acute basis.

Sexual Dysfunction and Sexual Performance

Erectile dysfunction is the inability to obtain and maintain a penile erection sufficient for satisfactory intercourse or other sexual expression. A number of factors can place an individual at risk for this disorder, for example, trauma, pelvic surgery, hypercholesterolemia, ischemic heart disease, peripheral vascular disease, chronic renal failure, diabetes, or the use of medicaments such as antihypertensive medication or digoxin, or illicit drugs, cigarettes or alcohol. Methods for the treatment of erectile dysfunction include but are not limited to: psychotherapy, the use of vacuum devices and penile implants, administration of medicaments such as yohimbine, papaverine and apomorphine, as well as treatment with phosphodiesterase-5 (PDE-5) inhibitors such as vardenafil, tadalafil, and sildenafil.

PDE-5 inhibitors enhance a man's ability to obtain and maintain erections. There are other drugs in clinical trials for treatment of erectile dysfunction that target other physiological pathways. For example, PT-141, from Palatin Technologies, targets the central nervous system. Endothelin antagonists are another class of compounds proposed for treatment of erectile dysfunction. The pharmacological treatments for erectile dysfunction are normally quite effective, but they do not cure the affliction or reverse the underlying problems; rather, they only have an acute, temporary benefit. By administering sub-antimicrobial DCS to a subject with erectile dysfunction in conjunction with a successful sexual outcome, the heightened confidence and reduced sexual performance anxiety resulting from a successful outcome can be consolidated in the subject's psyche, thereby facilitating extinction of any deleterious performance anxiety associated with sexual intercourse.

Accordingly, one embodiment of the methods of the invention entails administering sub-antimicrobial DCS to a male in conjunction with an extinction learning event, wherein an extinction learning event comprises a successful sexual outcome. In this case, DCS would be administered after the successful sexual outcome. In another embodiment of the invention, sub-antimicrobial DCS is administered within about eight hours, preferably within about four hours prior to, a psychotherapy session aimed at reducing anxiety related to a subject's sexual performance. Another embodiment of the methods of the invention entails administering sub-antimicrobial DCS to a male in conjunction with an extinction learning event resulting from administration of a PDE-5 inhibitor; i.e., one embodiment of the invention comprises:
(1) administering to a subject a pharmacologic agent known to alleviate erectile dysfunction or enhance sexual performance;
(2) administering to said subject a sub-antimicrobial dose of DCS; and
(3) a successful sexual outcome,
   wherein the DCS can be administered simultaneously with the pharmacological agent known to alleviate erectile dysfunction, or can be administered separately.

While the methods of the invention are useful for patients afflicted with erectile dysfunction, the methods of the invention do not require that the subject be afflicted with erectile dysfunction. In some embodiments of the invention, a pharmacologic agent useful for treating erectile dysfunction is administered to subjects because it improves, or is believed to improve, male sexual performance. As defined herein, a “successful sexual outcome” is one in which the subject perceives that he has had a positive sexual performance. For example, a man with erectile dysfunction may perceive a positive sexual performance as one in which he achieves and maintains an erection until orgasm. For other men, a successful sexual outcome might be one in which ejaculation is delayed for as long as the man would like.

In one embodiment of the invention, a subject undergoes a course of treatment ranging from one to ten pharmaceutical interventions comprising:
1. administering to the subject an efficacious PDE-5 inhibitor,
2. a successful sexual outcome, and
3. administering to the subject sub-antimicrobial DCS, within about eight hours prior to or four hours following the subject's sexual encounter.
   At the conclusion of this course of treatment, deleterious performance anxiety in a subject with erectile dysfunction should be substantially reduced or eliminated. Therefore and thereafter, the physiological boost (i.e., a PDE-5 inhibitor) required for successful sexual performance is reduced. For erectile dysfunction subjects for whom the etiology is primarily psychogenic, this removal of deleterious performance anxiety may be sufficient to cure the subject, eliminating the need for future pharmaceutical intervention. For erectile dysfunction subjects with significant physiological impediments to achieving or maintaining erections, pharmaceutical therapy may still be required; however, the success rate of that pharmaceutical therapy will be higher, as the physiological boost provided by the drug will no longer have to overcome the additional impediment of negative performance anxiety. In other words, even if it does not provide a cure, the combination of sub-antimicrobial DCS and one or more PDE-5 inhibitors can improve the efficacy of ongoing treatments by eliminating the negative influence of performance anxiety. Accordingly, the methods and compositions of the invention are useful for the treatment of most erectile dysfunction subjects, not limited to those subjects for whom the affliction is primarily psychogenic.

Many pharmacologic agents that have been approved or suggested for treatment of male erectile dysfunction have also been suggested or attempted as treatments for female sexual dysfunction. For example, the following agents have been tested, on either an acute or chronic basis, for treatment of female sexual dysfunction: alprostadil, phentolamine, estradiol, flibanserin, apomorphine, bupropion, testosterone, sildenafil, PT-141, vardenafil, yohimbine, tadalafil, and combinations thereof. These agents are sometimes administered orally, and in other cases are administered as creams, inhalation sprays, or transdermally. The present invention contemplates methods and compositions for alleviating female sexual dysfunction comprising administering to a female subject a combination of (i) one or more of the above-listed drugs, (ii) an extinction learning event, and (iii) administering to the subject a pharmacologic agent that enhances learning, preferably DCS. For females afflicted with female sexual dysfunction, an extinction learning event can be orgasm, arousal, or heightened sexual desire. Another embodiment of the methods of the invention entails administering sub-antimicrobial DCS to a female subject afflicted with female sexual dysfunction in conjunction with an extinction learning event for female sexual dysfunction. In this case, DCS would normally be administered after the extinction learning event, but would preferably be administered before the extinction learning event.

In preferred embodiments, sub-antimicrobial DCS is administered on an acute basis.

Compositions can be formulated in accordance with methods known in the art. For example, the following components are sieved and mixed intimately: DCS (200 g), sildenafil citrate (50 g), a mixture of lactose and microcrystalline cellulose (700 g), polyvinyl pyrrolidone (100 g), cellulose ether (100 g), silicon dioxide (4 g). Magnesium stearate is also sieved and admixed to the mixture of the other components. The composition is tableted directly to produce tablets containing 200 mg DCS and 50 mg sildenafil citrate. After pressing, the tablet core is coated with an aqueous film. The film thickness is variable.

In an exemplary embodiment, the following components are sieved and mixed intimately: DCS (50 g), sildenafil citrate (50 g), a mixture of lactose and microcrystalline cellulose (700 g), polyvinyl pyrrolidone (100 g), cellulose ether (100 g), silicon dioxide (4 g). Magnesium stearate is also sieved and admixed to the mixture of the other components. The composition is tableted directly to produce tablets containing 50 mg DCS and 50 mg sildenafil citrate. After pressing, the tablet core is coated with an aqueous film. The film thickness is variable. This tablet, containing a combination of sildenafil and a sub-antimicrobial dose of DCS, is useful according to the methods and compositions of the invention.

In another embodiment, a mixture of 50 g of DCS, 20 g tadalafil, 75 g of lactose and 100 g of talc is combined, mixed, wetted with a sufficient quantity of alcohol, and granulated followed by drying. The obtained granulate can be either pressed to form tablets or filled into capsules containing 50 mg DCS and 20 mg tadalafil. The pharmaceutical compositions can be useful for alleviating erectile dysfunction or enhancing sexual performance.

In another embodiment, a mixture of 100 g of DCS, 20 g vardenafil HCl, 75 g of lactose and 100 g of talc is combined, mixed, wetted with a sufficient quantity of alcohol and granulated followed by drying. The obtained granulate is either pressed to form tablets or filled into capsules containing 100 mg DCS and 20 mg vardenafil. The pharmaceutical compositions can be useful for alleviating erectile dysfunction or enhancing sexual performance.

In one embodiment of the invention, a subject seeking improved sexual performance ingests a sildenafil citrate tablet (50 mg) prior to a sexual encounter. After experiencing improved sexual performance, with attendant improved confidence, the subject is administered DCS (50 mg). The subject repeats this procedure on an approximately once-weekly basis for four weeks. After four weeks, the subject discontinues use of sildenafil and DCS prior to and following sexual relations. Relative to the subject's sexual performance prior to the four-week pharmacotherapy protocol, the subject's sexual performance after the four-week protocol is expected to improve as a result of enhanced confidence and reduced anxiety.

In another embodiment of the invention, a subject afflicted with female sexual dysfunction, due to a general lack of desire, engages in arousing foreplay and sexual relations. Immediately thereafter, the subject is administered DCS (75 mg). The subject continues to take DCS on an acute basis after extinction learning events that provide her with enhanced sexual desire in an attempt to consolidate learning pertaining to the increased sexual desire.

In another embodiment of the invention, a subject afflicted with female sexual dysfunction, resulting from an inability to reach orgasm consistently, is administered sildenafil (50 mg). After engaging in arousing foreplay and sexual relations, the subject experiences orgasm. The subject is administered sildenafil (50 mg) prior to some but not all sexual relations over a period of several months. After every romantic interlude resulting in orgasm, irrespective of whether or not sildenafil was administered, the subject is administered DCS (75 mg). After three months of this procedure, a responsive subject can experience an increase in the percentage of sexual encounters that result in orgasm.

In one embodiment of the invention, a clinical study with the following summarized protocol can be used to study the benefits provided by administering sub-antimicrobial doses of DCS in conjunction with sildenafil therapy for treatment of erectile dysfunction. Erectile dysfunction has been shown to be responsive to pharmacological treatment with PDE-5 inhibitors such as sildenafil. In the randomized, placebo-controlled clinical study described below, acute treatment with sub-antimicrobial DCS is utilized with the intent of enhancing the effects of treatment of erectile dysfunction with a PDE-5 inhibitor. There are three arms to the study: subjects taking placebo only, subjects taking a PDE-5 inhibitor only, and subjects taking a combination of a PDE-5 inhibitor and DCS. Specifically, combination therapy subjects are administered a capsule containing a combination of sildenafil citrate (50 mg) and DCS (75 mg). Mono-drug therapy subjects are administered a capsule containing sildenafil citrate (50 mg). Subjects are classed into three different categories (organic, psychogenic, mixed) based on etiologies of erectile dysfunction. All subjects are told that they will be in a clinical trial, with various arms, studying the ability of a new drug to alleviate erectile dysfunction. Analogous to previous clinical studies for erectile dysfunction, the effects of treatments are assessed for the men's abilities to engage in sexual activity and to achieve and maintain erections sufficient for satisfactory sexual activity. Subject self-assessment of sexual function, at baseline and during and after the study, is used to evaluate the success of the therapy. Two important end-point questions are frequency of successful penetration during sexual activity and maintenance of erections after penetration. After one month on the drug therapy, the drug therapy is ended, and the subjects are evaluated, using the same sexual function criteria, for the next three months, a period during which the subjects do not take drugs for erectile dysfunction.

Chronic Pain

Many individuals suffer from chronic pain, including long-standing neuropathic pain. Numerous non-pharmacologic techniques are used to treat chronic pain, including transcutaneous electrical nerve stimulation (TENS), acupuncture, physical therapy, massage, relaxation therapy, biofeedback, and psychotherapy, in addition to pharmacotherapy. Medications from several different drug classes are commonly used to treat chronic pain, including topical agents, tricyclic antidepressants, serotonin specific reuptake inhibitors (SSRIs), anticonvulsants, and nonopioid analgesics.

Recent studies (Wager et al. (2004), Science 303: 1162-1167) have demonstrated that people experience pain differently when they believe that the pain will be alleviated. The experience of pain arises from both physiological and psychological factors, including one's beliefs and expectations. Thus, placebo treatments that have no intrinsic pharmacological effects may produce analgesia by altering expectations. In two functional magnetic resonance imaging (fMRI) experiments, researchers found that placebo analgesia was related to decreased brain activity in pain-sensitive brain regions, including the thalamus, insula, and anterior cingulate cortex, and was associated with increased activity during anticipation of pain in the prefrontal cortex, providing evidence that placebos alter the experience of pain.

Given this result, it is clear that a subject's response to painful stimuli is governed by a number of factors, many of which are psychological. If a subject is anxious about the pain, the pain that is experienced in normally worse than if the subject is not anxious about the pain. As a result, chronic pain has been treated effectively using cognitive behavioral therapy. The methods of the invention aim to reduce the psychological component associated with chronic pain. One method to do so would be to render permanent a subject's temporal, low-anxiety response to chronic pain. By administering sub-antimicrobial DCS to a subject in conjunction with extinction training, a beneficial, low-anxiety response to chronic pain can be consolidated and rendered more likely to be repeated in the future. Accordingly, administering sub-antimicrobial DCS to subjects suffering from chronic pain can be particularly beneficial according to the methods of the invention by administering DCS within eight hours prior to cognitive behavioral therapy or biofeedback therapy, or within four hours following completion of a cognitive behavioral therapy session or a biofeedback therapy session.

In preferred embodiments, sub-antimicrobial DCS is administered on an acute basis.

Insomnia

Extinction training for reducing anxiety associated with insomnia entails subjecting a subject afflicted with insomnia to an environment wherein a stimulus is presented that frequently generates a deleterious, high-anxiety response (e.g., an attempt to fall asleep), but conditions are controlled to reduce anxiety or produce a favorable outcome (i.e., falling asleep relatively easily), or both. According to the methods of the invention, sub-antimicrobial DCS is administered to a subject within eight hours, preferably within four hours, of the extinction learning event.

Psychotherapy, biofeedback training, and acupuncture are all non-pharmaceutical methods for treating insomnia. Any of these methods can be combined with administering sub-antimicrobial DCS according to the methods of the invention.

Pharmacologic agents useful for treatment of insomnia can be also used in the methods of this invention. Zaleplon, zopiclone, eszopiclone, indiplon, and zolpidem are all central nervous system depressants useful for treatment of insomnia. Benzodiazepines, e.g., lorazepam, clonazepam, oxazepam, flurazepam, triazolam, temazepam, alprazolam, and pharmaceutically acceptable salts thereof, are also frequently used to treat insomnia, although it should be noted that benzodiazepines may interfere with the extinction process, and therefore counter the action of DCS. By taking a drug that is likely to induce sleep, a subject suffering from insomnia will be more likely to have a positive outcome (i.e., falling asleep) upon exposure to a stimulus (i.e., going to bed and attempting to fall asleep) that often generates substantial anxiety in the subject. The positive outcome, and reduced anxiety, will be consolidated as a new learning by administering sub-antimicrobial DCS in conjunction with a suitable sleeping aid. Accordingly, pharmaceutical combinations of (i) sub-antimicrobial DCS, and (ii) one or more pharmacologic agents useful for treatment of insomnia, are contemplated according to the methods and compositions of the present invention. In some embodiments, subjects can be co-administered sub-antimicrobial DCS and one or more pharmacologic agents generally known to be useful for treatment of insomnia, including but not limited to zaleplon (between 5 mg and 40 mg), zopiclone (between 2.5 mg and 50 mg), zolpidem (between 2.5 mg and 40 mg), eszopiclone (between 1 mg and 10 mg), and indiplon (between about 2.5 mg and 50 mg), and pharmaceutically acceptable salts thereof.

In preferred embodiments, sub-antimicrobial DCS is administered on an acute basis.

Compositions that can be utilized according to the methods of the invention can be formulated in accordance with methods known in the art. For example, in one embodiment, a mixture of 50 g of DCS, 5 g of zolpidem tartrate, 50 g of lactose and 75 g of talc is combined, mixed, wetted with a sufficient quantity of alcohol and granulated followed by drying. The obtained granulate is either pressed to form tablets or filled into capsules containing 50 mg DCS and 5 mg zolpidem tartrate. The pharmaceutical compositions can be useful for alleviating insomnia.

In another embodiment, tablets containing amitriptyline (between 10 and 200 mg) and DCS (75 mg) are formed from standard pharmaceutical excipients using standard technology for forming tablets. The tablets can be useful for alleviating insomnia.

In one embodiment of the invention, a clinical study with the following summarized protocol can be used to study the benefits provided by administering sub-antimicrobial doses of DCS in conjunction with zolpidem therapy for treatment of insomnia. Insomnia has been shown to be responsive to pharmacological treatment with compounds such as zolpidem tartrate. In the randomized, placebo-controlled clinical study described below, acute treatment with sub-antimicrobial DCS is utilized with the intent of enhancing the effects of treatment of chronic insomnia with zolpidem tartrate. There are three arms to the study: subjects taking placebo only, subjects taking only zolpidem tartrate (10 mg daily), and subjects taking a combination of zolpidem tartrate and DCS. Specifically, combination therapy subjects are treated via daily administration of one of two types of pharmaceuticals. On days 2-7,9-14, 16-21, and 23-28, the subjects ingest a capsule containing zolpidem tartrate (10 mg) as the only active ingredient. On days 1, 8, 15, and 29, subjects ingest a capsule containing both zolpidem tartrate (10 mg) and DCS (50 mg). All subjects are told that they will be in a clinical trial, with various arms, studying the ability of a new drug to alleviate insomnia. Sleep latency and sleep efficiency are evaluated in the subjects. Subject self-assessment of insomnia is also used to evaluate the success of the therapy. A follow-up evaluation of subjects is conducted after three months. Endpoint questions include frequency of insomnia and duration of insomnia.

In another embodiment of the invention, a subject suffering from chronic insomnia is treated on a daily basis for 30 days by being administered two pharmacologic agents:
(1) a coated tablet containing either DCS (100 mg) or placebo; and
(2) indiplon, 20 mg
   The first pharmacologic agent (i.e., the coated table containing DCS or placebo) is provided in a pharmaceutical kit comprising a serially numbered package, with DCS tablets corresponding to days 3, 10, 17, and 24; and placebo tablets on all other days. The placebo tablets and DCS tablets are visually indistinguishable. Relative to subjects treated with indiplon alone, the subject receiving the combination therapy may be less likely to experience a recurrence of the condition that is sufficient to require additional drug therapy.

In another embodiment, a subject suffering from chronic pain and sleeping difficulties is administered amitriptyline (20 mg) on a daily basis for 30 days. Every seven days, the subject is additionally treated with DCS (50 mg). Relative to subjects treated with amitriptyline alone, the subject treated additionally with DCS on an acute basis may experience a more positive pharmacotherapy outcome, resulting in an increased alleviation of chronic pain and/or sleeping problems.

Chronic Fatigue Syndrome, Excessive Daytime Sleepiness, and other Disorders

The methods and compositions of the invention are useful for treating other medical afflictions that are exacerbated by anxiety about the affliction. Some, but not all, individuals afflicted with chronic fatigue syndrome, excessive daytime sleepiness, attention deficit-hyperactivity disorder, and eating disorders can benefit from the methods of the invention. Extinction training for reducing anxiety associated with these afflictions varies according to the affliction and the subject. In general, it entails subjecting a subject afflicted with the disorder to an environment wherein a stimulus is presented that frequently generates a deleterious, high-anxiety response, but conditions are controlled to reduce anxiety or produce a favorable outcome. In particular, a combination of modafinil and DCS may be useful to treat individuals afflicted with chronic fatigue syndrome and excessive daytime sleepiness. In preferred embodiments of the invention, modafinil may be administered on a chronic or acute basis, and DCS is administered on an acute basis.

In one embodiment of the invention, DCS (50 g), modafinil (200 g), a mixture of lactose and microcrystalline cellulose (700 g), polyvinyl pyrrolidone (100 g), cellulose ether (100 g), and silicon dioxide (4 g) are sieved and mixed intimately. The composition is tableted directly to produce tablets containing 50 mg DCS and 200 mg modafinil. After pressing, the tablet core is coated with an aqueous film.

In an embodiment of the invention, a subject suffering from excessive daytime sleepiness is treated daily for 30 days with pharmaceutical tablets arranged in a serially numbered package comprising tablets of the following two types:
(1) modafinil (100 mg) and DCS (50 mg)
(2) modafinil (100 mg)
   The two types of tablets are indistinguishable. The combination tablets containing both modafinil and DCS correspond to days 3, 9, 15, 21, and 27 in the numbered package, with modafinil tablets (not containing DCS) provided for all other days in the pharmaceutical kit. Relative to subjects treated with modafinil alone, a subject receiving the combination therapy may experience a greater decrease in excessive daytime sleepiness if the subject has anxiety about the condition that is relieved by the pharmacological benefits provided by modafinil.

In another embodiment of the invention, a subject suffering from chronic fatigue syndrome is administered modafinil (200 mg) on a daily basis for 30 days. Every seven days, the subject is additionally treated with DCS (50 mg). Relative to subjects treated with modafinil alone, a subject receiving the combination therapy may experience a greater decrease in chronic fatigue syndrome if the subject has anxiety about the condition that is alleviated by the pharmacological benefits provided by modafinil.

In another embodiment of the invention, a subject suffering from chronic fatigue syndrome is administered adrafinil (300 mg) on a twice-daily basis for 30 days. On days when the subject feels particularly energetic, the subject is additionally treated with DCS (50 mg). Relative to subjects treated with adrafinil alone, the subject receiving co-administered DCS may experience a more positive pharmacotherapy outcome, resulting in an increased alleviation of excessive daytime sleepiness.

In another embodiment, a subject suffering from excessive daytime sleepiness is administered a tablet containing modafinil (100 mg) and DCS (100 mg) on days when said subject is feeling particularly tired. The subject repeats this behavior on other days when he is feeling particularly drowsy, provided that said subject has not taken DCS on the previous day, or preferably within the previous three days. Relative to subjects treated with modafinil on a daily basis, the subject treated on an acute basis with a combination of modafinil and DCS may experience an enhanced alleviation of excessive daytime sleepiness after completion of the pharmaceutical therapy, particularly as measured months after completion of the therapy.

In another embodiment, a student unnerved by insufficient studying during a school term is concerned that by studying for numerous hours late at night, he will be less efficient on an exam the following day. The student feels he needs to do more studying, but is worried about the diminished value of studying late at night, due to decreased retention of knowledge. Moreover, the student is concerned about poor mental acuity during the exam the following day as a result of insufficient sleep. In order to increase alertness during the study period and improve learning retention, the student ingests a tablet containing modafinil (100 mg) and DCS (80 mg).

In another embodiment, a juvenile subject afflicted with attention deficit-hyperactivity disorder is administered methylphenidate (20 mg) on a twice-daily basis. The pharmaceutical therapy has been continued for three years, with modest increases in dose. The subject and his parents recognize that in spite of taking methylphenidate on a daily basis, the severity of the symptoms of the affliction varies on a daily basis, and even on an hourly basis. The subject's parents have noted that the subject generally has a high relative ability to maintain attention after long periods of exercise. Accordingly, the subject is administered DCS (25 mg) on an approximately once-weekly basis prior to commencing a lengthy and rigorous bike ride.

Animal Training

In some embodiments of the invention, the subject is a mammal other than a human. In some preferred embodiments, the subject is a dog, and sub-antimicrobial DCS is administered to the dog in conjunction with extinction training. Suitable forms of extinction training include but are not limited to: training to reduce separation anxiety, extinction training to reduce anxiety associated with a particular noise (e.g., thunderstorm), training for obedience skills, and training to reduce destructive behavior.

In one embodiment of the invention, a 6-month old Chesapeake Bay Retriever dog (weighing approximately 40 pounds) undergoes an extensive training session, including socialization with other dogs and people, during which she repeatedly responds to familiar and unfamiliar obedience commands. At the outset of the session, and again at the end of the session, the dog is administered approximately 10 mg of DCS (mixed with a spoonful of peanut butter). It is anticipated that the positive behavioral effects of the training will be enhanced by the administering of DCS.

In another embodiment of the invention, an 18-month old Chesapeake Bay Retriever dog (weighing approximately 80 pounds) is fearful of entering a local dog park, having been unexpectedly attacked and bitten by another dog on her first visit to the dog park. She visits the dog park one time per week over a 10-week span, and each time is administered approximately 30 mg of DCS (mixed with peanut butter) within one hour prior to entering the dog park. Her anxiety level is then assessed after 10 weeks of this procedure, to determine if she has reasonably extinguished her fear regarding entering the dog park.

A subject undergoing treatment with the methods of the invention can experience improved extinction of the deleterious, high-anxiety response that the treatment is intended to eliminate. This facilitated extinction is manifested as reduced anxiety upon exposure to a stimulus that previously prompted the deleterious, high-anxiety response. This reduction in anxiety can lead to improvement in one or more symptoms associated with the various afflictions that can be treated according to the methods of the invention. In preferred embodiments, sub-antimicrobial DCS is administered on an acute basis.

The efficacy of the methods of the invention can be assessed using any clinically recognized assessment method for measuring a reduction of one or more symptoms of the particular anxiety disorder or other afflictions that are treated. It should be noted that not every subject will benefit from the methods of the invention, just as other pharmaceutical agents do not typically benefit every patient.

Formulation of Pharmaceutical Compositions

Pharmaceutical compositions contemplated by the methods and compositions of the invention may be formulated and administered to a subject for treatment of the diseases or afflictions disclosed herein as described below.

The invention encompasses the preparation and use of pharmaceutical compositions comprising sub-antimicrobial DCS (or DCS in combination with another active agent) as an active ingredient useful for treatment of the afflictions disclosed herein. Such pharmaceutical compositions may consist of sub-antimicrobial DCS alone, in a form suitable for administration to a subject, or the pharmaceutical composition may comprise DCS and one or more pharmaceutically acceptable carriers, one or more additional ingredients, or some combination of these. The active ingredient(s) may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

The descriptions of pharmaceutical compositions provided herein are directed to pharmaceutical compositions which are suitable for ethical administration to humans and other mammals.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations. Preferably, the formulations are suitable for oral administration.

The relative amounts of the active ingredient, the pharmaceutically acceptable carrier, and any additional ingredients in a pharmaceutical composition of the invention may vary, depending upon the identity, size, and condition of the subject treated and further depending upon the route by which the composition is to be administered. By way of example, the composition may comprise between 0.1% and 100% (w/w) active ingredient.

The therapeutically effective dose of the pharmacologic agent can be administered using any medically acceptable mode of administration. Although the skilled artisan would contemplate any of the modes of administration known to one of ordinary skill, preferably the pharmacologic agent is administered according to the recommended mode of administration, for example, the mode of administration listed on the package insert of a commercially available agent.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion. As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

DCS has been reported to reduce the levels of certain important chemicals in the blood of subjects, including calcium, folic acid, magnesium, vitamin K, vitamin B6, and vitamin B12. Co-administration of DCS with supplements of any of these ingredients is contemplated by the methods and compositions of this invention. In particular, supplementation of DCS with vitamin B6, vitamin B12, or combinations thereof is contemplated. In one embodiment, a tablet containing 50 mg DCS also contains 50 mg pyridoxine. In other embodiments of the invention, pyridoxine is supplemented at levels up to ten times the dosage of DCS.

Controlled-Release Formulations

Controlled-release formulations of DCS pharmaceutical composition useful in the methods and compositions of the invention may be made using techniques known in the art. In the present context, a “controlled-release” formulation is one that substantially alters the absorbance of the active ingredient in the body relative to a simple oral composition containing the same ingredients. More specifically, a controlled release formulation is one wherein for a given amount X (mg) of active ingredient Y, the serum concentration B_{cr} (μg/mL) of active ingredient Y at a given time T (minutes after oral administration) in a human subject, resulting from administration of a controlled-release formulation of Y in said human subject, will differ by more than a factor of two from the serum concentration Bₛₜ of active ingredient Y at time T in said human subject resulting from administration of a standard oral formulation of Y containing the same amount X of the active ingredient Y, and wherein time T is greater than 60 minutes.

The benefits of incorporating DCS itself into a controlled-release formulation are likely to be only modest, and in some embodiments non-existent, when used according to the methods of the present invention. That said, in some embodiments of the invention, DCS is combined with other active agents, and the additional benefit of incorporating the other active agent into a controlled-release formulation may be substantial.

The methods and compositions of this invention contemplate many different controlled-release pharmaceutical compositions and combinations, with varying pharmacokinetics. A pharmaceutical composition combining two compounds may contain one compound that is in an controlled-release dosage form, while the other compound is not formulated for controlled release.

One of the most frequently utilized methods to extend the duration of drug action in the body is by modification of the pharmaceutical dosage form. This is usually achieved with single or multi-component matrix systems such as granules, pellets, tablets or a combination of the above where the drug delivery is mainly controlled by diffusion or erosion mechanisms.

Another commonly used procedure to sustain or control the rate of drug release is by utilizing polymer coating technology. Polymers with pH-dependent or pH-independent properties are coated onto the different dosage forms utilizing fluid bed or conventional coating equipment. Other methods known in the art, including liposome formation, may also be used to provide sustained release according to the methods of the invention.

The composition according to the invention may be in the form of granulates, pellets, spheroids and/or extrudates. These may either be filled into capsules or sachets or pressed to form tablets. Moreover, the active ingredient and possible additives may optionally be tabletted directly.

The active ingredients used in the methods and compositions of the invention may be embedded in a matrix that ensures the sustained release of the active ingredients over a period of at least 8 to 12 hours and optionally of up to 24 hours (matrix-controlled). Suitable matrix-forming materials include but are not limited to: a) hydrophilic or hydrophobic polymers; b) digestible, substituted or unsubstituted long-chain (C₈-C₅₀) hydrocarbons; c) polyalkylene glycols, wherein the composition according to the invention may contain up to 60% (weight percent) of one or more polyalkylene glycols, based on the matrix.

The active ingredients may be incorporated in a gel-forming matrix of, e.g., hydroxymethyl cellulose, hydroxyethyl cellulose, hydroxypropyl cellulose, hydroxypropylmethyl cellulose, alginate and/or polyacrylic acid, in particular hydroxypropylmethyl cellulose. The polymer hydrates to form a gel-like layer, i.e., a hydrogel matrix that slowly releases the active ingredient in a controlled manner by way of diffusion and erosion.

In another matrix form according to the invention, the active ingredients may be combined with known water-soluble additives and fatlike substances. As lipophilic substances degradable mono-, di- and triglycerides (glycerol monostearate, glycerol monooleate, glycerol tripalmitate), but also erodable fatty alcohols (lauric, myristic, stearic, cetylic and/or ceryl alcohol) having a melting point in the range of from 30-80° C. may be used. Delivery of the active ingredient takes place by diffusion and by enzymatic degradation of the lipophilic substances. Embedding of the active ingredient into the matrix is achieved by melting, spray solidification, spray-drying, granulating or direct tabletting.

A further useful controlled-release matrix form may contain, in addition to the active ingredients, known water-soluble additives which are embedded, just like the active ingredient, in a framework structure formed of water-insoluble, indigestible additives. Elution of the soluble constituents generates pores through which the active ingredient diffuses to the outside. Polymers such as polyvinyl chloride, polyethylene, polyamide, silicones, ethyl cellulose and/or methacrylate-acrylate copolymers may be employed as structure-building substances. The mixture of active ingredient/additive is either immediately pressed to form tablets or following wet granulation with organic solvents or binder solutions, or it is filled into capsules in pellet form.

The controlled-release matrix may contain further pharmaceutically useful additives which are conventional according to the prior art, such as, e.g., diluents, lubricants, binders, granulation aids, colorants, flavoring agents, detergents, buffers, anti-blocking agents and/or lubricating agents.

Another embodiment of the invention may consist of an initial dose and a delayed-release component. The initial dose may contain the active ingredients as a powder, granulate and/or pellets, optionally together with respective additives. One or more active ingredients contained in the initial dose are released immediately following administration. The therapeutically effective blood plasma level is attained very rapidly by means of this initial dose, so that a therapeutic effect is observed shortly after administration. The delayed-release component contains one or more active ingredients incorporated within a suitable matrix, as described above.

For the preparation of the above mentioned dosage forms, the pharmaceutical additives known from the prior art may be used, such as tablet binders, fillers, preservatives, tablet disintegrants, flow regulating agents, plasticizers, wetting agents, dispersing agents, emulsifiers, retarding agents, antioxidants and/or other known carrier substances and diluents.

### EXAMPLES

The invention is now described with reference to the following examples. These examples are provided for the purpose of illustration only and the invention should in no way be construed as being limited to these examples but rather should be construed to encompass any and all variations which become evident as a result of the teaching provided herein.

All FDA-approved pharmacologic agents used in the following examples were obtained from commercial suppliers and/or pharmacies. All active ingredients used in human subjects in the following examples have been approved for use in humans by the FDA. Capsules containing mixtures of multiple active ingredients were obtained by measuring out solid powders or granulates of the active ingredients and loading them into the capsules.

Examples 1-6 were conducted to examine the effects of the partial NMDA receptor agonist DCS on conditioned fear extinction. These experiments were conducted using adult male Sprague-Dawley rats as described below. Examples 7 and 8 describe a clinical trial of DCS augmentation of behavioral exposure therapy for human subjects suffering from a specific phobia. Example 9 describes the use of DCS in a subject to treat chronic pain. Example 10 describes use of DCS in a subject, in conjunction with zolpidem tartrate, to treat insomnia. Examples 11 and 12 describe methods and compositions administered to a control subject that would be useful to treat sexual dysfunction or otherwise enhance sexual performance.

Materials and Methods for Examples 1-6

Animals

Adult male Sprague-Dawley rats (Charles River, Raleigh, N.C.) weighing between 300 and 400 g were used. Animals were housed in group cages of four rats each in a temperature (24° C.) controlled animal colony, with continuous access to food and water. They were maintained on a 12:12 light-dark cycle with lights on at 0700 hours. All behavioral procedures took place during the rats' light cycle. A total of 178 rats were used.

Apparatus

Animals were trained and tested in 8×15×15-cm Plexiglas and wire mesh cages. The cage floor consisted of four 6.0-mm diameter stainless steel bars spaced 18 mm apart. Each cage was suspended between compression springs within a steel frame and located within a custom-designed 90×70×70-cm ventilated sound-attenuating chamber. Background noise (60 dB wide-band) was provided by a General Radio Type 1390-B noise generator (Concord, Mass.) and delivered through high frequency speakers (Radio Shack Supertweeter; Tandy, Fort Worth, Tex.) located 5 cm from the front of each cage. Sound level measurements (SPL) were made with a Bruel & Kjaer (Marlborough, Mass.) model 2235 sound-level meter (A scale; random input) with the microphone (Type 4176) located 7 cm from the center of the speaker (approximating the distance of the rat's ear from the speaker).

Startle responses were evoked by 50-ms 95-dB white-noise bursts (5 ms rise-decay) generated by a Macintosh G3 computer soundfile (0-22 kHz), amplified by a Radio Shack amplifier (100 Watt; Model MPA-200; Tandy, Fort Worth, Tex.), and delivered through the same speakers used to provide background noise. An accelerometer (model U321AO2; PCB Piezotronics, Depew, N.Y.) affixed to the bottom of each cage produced a voltage output proportional to the velocity of cage movement. This output was amplified (PCB Piezotronics, Model 483B21) and digitized on a scale of 0-2500 units by an InstruNET device (GW Instruments, Model 100B; Somerville, Mass.) interfaced to a Macintosh G3 computer. Startle amplitude was defined as the maximal peak-to-peak voltage that occurred during the first 200 ms after onset of the startle-eliciting stimulus.

The CS was a 3.7-s light (82 lux) produced by an 8-W fluorescent bulb (100-μs rise time) located 10 cm behind each cage. Luminosity was measured using a VWR light meter (Atlanta, Ga.). The unconditioned stimulus was a 0.5-s shock, delivered to the floorbars, and produced by a LeHigh Valley shock generator (SGS-004; LeHigh Valley, Beltsville, Md.). Shock intensities (measured as in Cassella et al. (1986) Physiol. Behav. 36:1187-1191) were 0.4 mA. The presentation and sequencing of all stimuli were under the control of the Macintosh G3 computer using custom-designed software (The Experimenter, Glassbeads Inc.; Newton, Conn.).

Surgery and Histology

Rats that were to receive intra-amygdala infusions (Example 6) were anesthetized with Nembutal (sodium pentobarbital, 50 mg/kg, i.p) and placed in a stereotaxic frame (ASI Instruments, Inc., Warren, Mich.). The skull was exposed and 22-gauge guide cannulae (model C313G, Plastics One, Inc.; Roanoke, Va.) were implanted bilaterally into the basolateral nucleus of the amygdala (AP=−2.8; DV=−9.0; ML=+5.0 from bregma). Dummy Cannulae (model C313DC, Plastics One, Inc.) were inserted into each cannula to prevent clogging. These extended approximately 1 mm past the end of the guide cannula. Screws were anchored to the skull and the assembly was cemented in place using dental cement (The Hygenic Corp., Akron, Ohio).

Behavioral procedures began either 10 or 11 days after surgery. Cannulated rats subsequently received a chloral hydrate overdose and were perfused intracardially with 0.9% saline followed by 10% formalin. The brains were removed and immersed in a 30% sucrose-formalin solution for at least 3 days, after which 40-μm coronal sections were cut through the area of interest. Every fourth section was mounted and stained with cresyl violet.

Drug Administration

Systemic administration: DCS (Sigma-Aldrich, St. Louis, Mo.)—(3.25, 15, and 30 mg/kg)—and (±)-HA-966 (Research Biochemicals, Inc., Natick, Mass.)—(6 mg/kg) were freshly dissolved in saline and injected intraperitoneally 30 minutes prior to extinction training.

Intra-Amygdala Infusion: DCS (10 μg/side) or saline was infused (0.25 μl/min) through 28-gauge injection cannulas (model C3131, Plastic Products) 20 min prior to extinction training. The total volume infused was 0.5 μl/side. The infusion cannulae were left in place for 2 minutes before being withdrawn.

General Behavioral Procedures

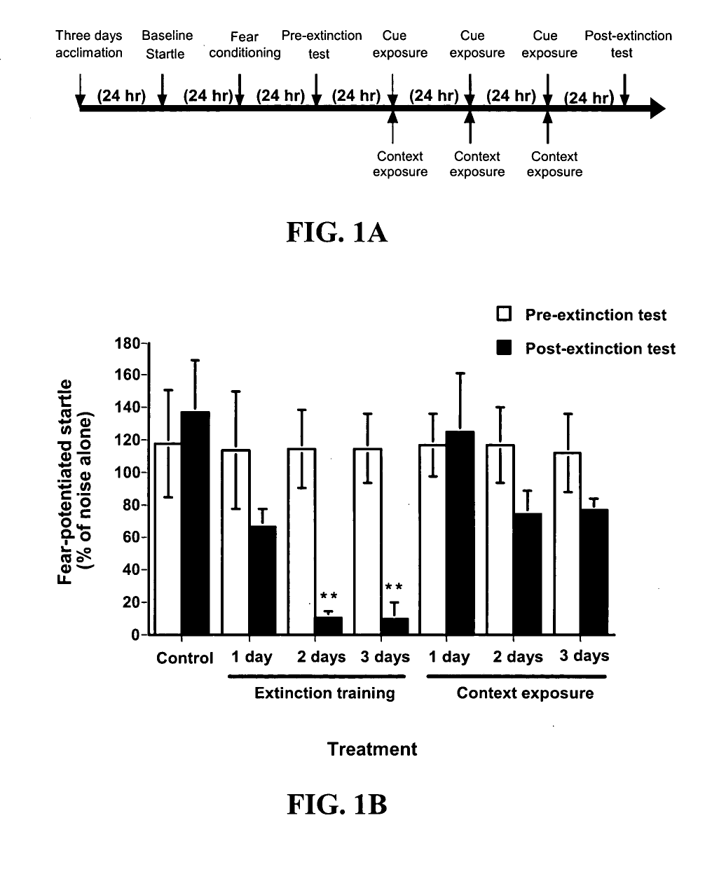

Behavioral procedures for all experiments consisted of an acclimation phase, a baseline startle test, a fear conditioning phase, a pre-extinction test, extinction training, and a post-extinction test (see FIG. 1A). The basic neuroscience behind this invention uses an animal paradigm called fear-potentiated startle. In this test, a rat is first conditioned to be afraid of a light by pairing it with a mild shock to its feet. The rat is then tested by eliciting a startle response with a loud sound in the presence or absence of the light. Fear-potentiated startle is defined as greater acoustic startle amplitude in the presence vs. the absence of the light. The increased startle is an example of how a state of fear facilitates reflexes. Extinction involves presenting the light over and over again in the absence of shock. This results in a decrease in the magnitude of fear-potentiated startle.

Acclimation. On each of three consecutive days, rats were placed into the test chambers for 10 min and then returned to their home cages.

Baseline startle test On each of the next two consecutive days, animals were placed in the test chambers and presented with 30 95-dB noise bursts at a 30-s interstimulus interval (ISI). Animals whose baseline startle was less than 1% of the possible accelerometer output were excluded insofar as fear-potentiated startle cannot be properly measured with such a low baseline (a total of 2 rats out of 144 were excluded on this basis).

Fear conditioning. 24 hours later, rats were returned to the test chambers and 5 minutes later given the first of 10 light-footshock pairings. The 0.4-mA 0.5-s shock was delivered during the last 0.5 seconds of the 3.7-second light. The average inter-trial interval was 4 minutes (range=3-5 minutes).

Pre-extinction test. 24 hours after fear conditioning, rats were returned to the test chambers and five minutes later were presented with 30 95-dB noise bursts (30-s ISI). These initial startle stimuli were used to habituate the startle response to a stable baseline prior to the noise alone and light-noise test trials that followed. A stable baseline, in turn, reduces variability in the fear-potentiated startle measure described below. Thirty seconds later, twenty additional noise bursts were presented (ISI=30 s). Half of these were presented in darkness (noise alone test trial), and half were presented 3.2 seconds after onset of the 3.7-second light (light-noise test trial). The order of these two trial types was randomized with the constraint that no two trial types occurred more than twice in a row. Percent fear-potentiated startle was computed as [(startle amplitude on light-noise minus noise-alone trials)/noise-alone trials]×100. Based on these data, rats were sorted into equal size groups such that each group had comparable mean levels of percent fear-potentiated startle. Because the fear-potentiated startle test is itself an extinction procedure (i.e., CS presentations without shock), and because we wanted to minimize any incidental extinction prior to explicit extinction training with drug, a minimal number of CS presentations was used in this test compared to the more lengthy post-extinction test described below. We have found, however, that this abbreviated test is adequate for matching rats into different groups with comparable levels of fear-potentiated startle.

Extinction training. 24 hours after the pre-extinction test, rats were returned to the test chamber and five minutes later received 30 3.7-s light exposures without shock (ISI=30 s). Control rats were placed in the test cages and remained there for the same amount of time as rats in the extinction groups, but did not receive non-reinforced CS presentations. Rats in Example 1 received either one, two, or three sessions of extinction training with a 24-hour interval between each. Rats in all other examples received a single session of extinction training.

Post-extinction test. 24 hours after the last extinction session, rats were returned to the test chamber and, five minutes later, were presented with 30 95-dB noise bursts, as in the pre-extinction short-test, to habituate the startle response to a stable baseline prior to the noise alone and light-noise test trials that followed. Thirty seconds later, 60 inter-mixed noise alone and light-noise test trials (95 dB, ISI=30 s) were presented. Percent potentiated startle was calculated from the noise alone and light-noise test trials as previously described.

Statistics

ANOVA on percent fear-potentiated startle scores was the primary statistical measure. Comparisons between groups were also made using two-tailed t-tests for independent samples. The criterion for significance for all comparisons was p<0.05.

Results—Examples 1-6

### Example 1

### Parametric Evaluation of Different Amounts of Extinction Training

This example assessed the effect on fear-potentiated startle of one, two, or three days of extinction training. 42 rats were matched into 7 groups of 6 animals each based on their level of fear-potentiated startle in the pre-extinction test. Beginning 24 hours after the pre-extinction test, rats received one, two, or three consecutive days of extinction training (30 non-reinforced light presentations per day), or one, two, or three days of exposure to the context without extinction training. An additional control group was tested two days after the pre-extinction test without intervening exposures to either context or the visual CS.

FIG. 1B shows that after one day of extinction training, fear-potentiated startle was reduced by approximately 35% compared to the pre-extinction test. After two or three days, fear-potentiated startle was reduced by approximately 90%. A two-way ANOVA with Treatment (non-reinforced CS presentations versus context exposure alone) and Days (one, two, or three extinction sessions) as between-subjects factors indicated a significant Treatment effect, F(1, 30)=13.01, and also a significant Treatment X Days interaction, F(2, 30)=8.90. Thus, the reduction of fear-potentiated startle across days was greater in the groups that received non-reinforced CS exposures than in the groups that received context exposure alone. Individual comparisons between non-reinforced CS presentation and context-exposure groups indicated significant differences after two (t(10)=3.41) days and after three (t(10)=6.37) days. Significant differences versus the non-exposed control group were found versus rats that received one (t(10)=2.30), two (t(10)=4.33), or three (t(10)=4.26) days of extinction training.

### Example 2

### Dose-Response Function for the Effect of DCS on Extinction

Twenty-seven rats were acclimated, tested for baseline startle, fear-conditioned, and tested for fear-potentiated startle as previously described. Rats were then divided into four groups of seven animals each (except the DCS 30 mg/kg group where N=6] based on their pre-extinction level of fear-potentiated startle. 24 hours later, each rat was injected with either saline or DCS (3.25, 15, or 30 mg/kg; i.p.). Thirty minutes later, rats received a single session of extinction training. A single extinction session was used because the results of Example 1 indicated that this produced a minimal amount of extinction against which a facilitatory effect of DCS could be detected. Twenty-four hours later, rats were tested for fear-potentiated startle without drug injections in order to evaluate the effect on extinction of the previous drug treatments.

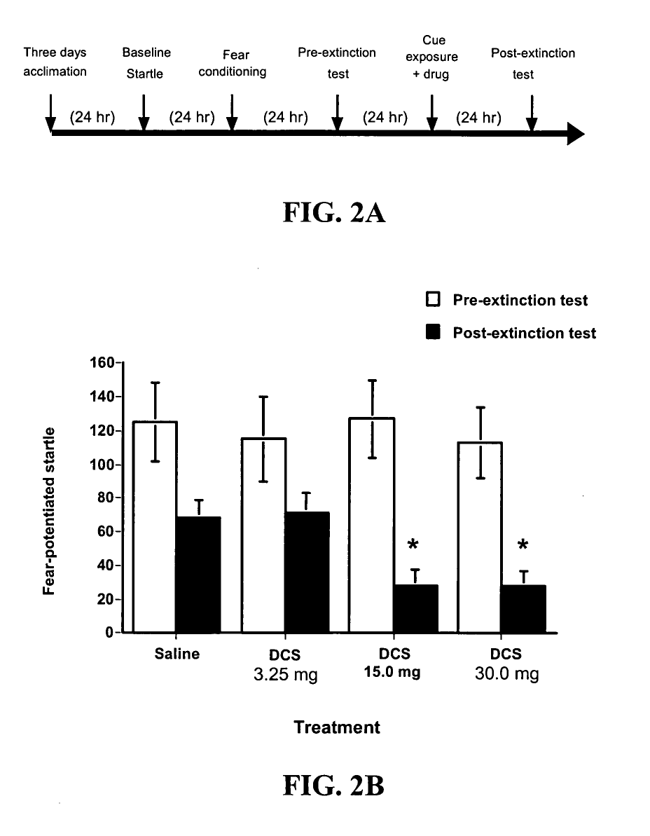

DCS facilitated extinction in a dose-dependent manner (FIG. 2B). ANOVA indicated a significant Dose effect, F(3,23)=3.02, with a significant linear trend, F(1,23)=7.26. Fear-potentiated startle was significantly lower in rats injected with 15 and 30 mg/kg DCS prior to extinction training, t(12)=2.61 and t(11)=2.53, for 15 and 30 mg/kg versus saline, respectively.

### Example 3

### Effect of DCS in Non-Extinguished Rats

To test whether the effects of DCS reflected an augmentation of extinction per se, or reflected, instead, a disruption of fear-potentiated startle independent of extinction (e.g., a delayed effect on the expression of fear-potentiated startle 24 hours after drug administration), additional rats were tested with and without extinction training. For this example, 28 rats were matched into four groups of seven animals each based on the pre-test. 24 hours later, each rat was injected with either saline or DCS (15 mg/kg) and returned to its home cage until placed in the startle chamber 30 minutes later. Two groups (one group of saline-injected rats and one group of DCS-injected rats) underwent extinction training. Two other groups (one group of saline-injected rats and one group of DCS-injected rats) were placed into the test chamber but did not receive extinction training. 24 hours later, all groups were tested for fear-potentiated startle without drug injections.

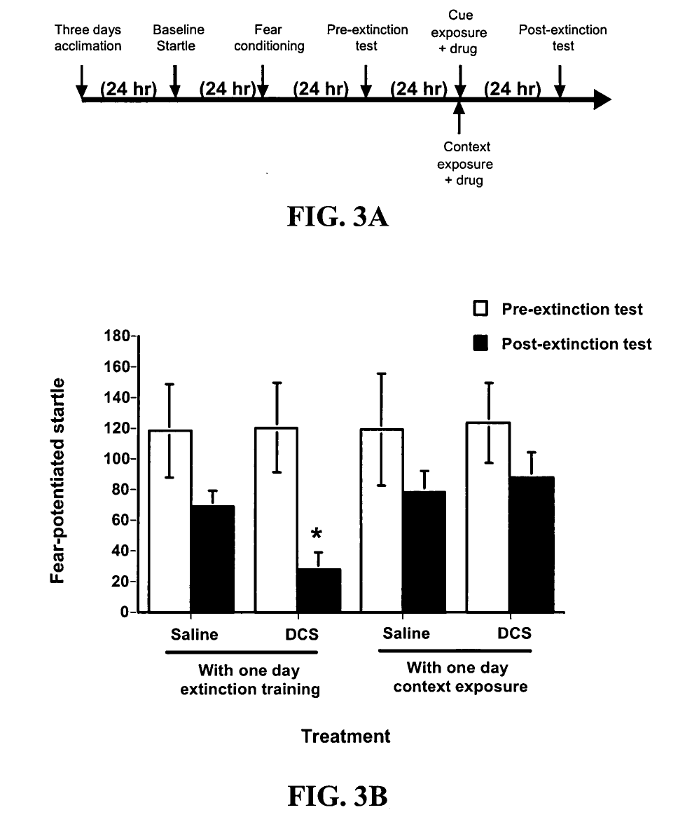

FIG. 3B shows that fear-potentiated startle in rats receiving DCS plus extinction training was significantly lower than in rats that received saline plus extinction training, t(12)=3.02. This replicates the principal finding of Example 2. The novel finding here is that fear-potentiated startle in rats that received DCS without extinction training was comparable to fear-potentiated startle in rats that received saline without extinction training. Thus, the effect of DCS noted in Example 2, and replicated here, appears to reflect a specific influence on extinction and not a more general effect on fear-potentiated startle measured 24 hours later in the absence of the drug.

### Example 4

### Effect of the Strychnine-Insensitive Glycine Recognition Site Antagonist, HA-966, on Extinction and on the Facilitation of Extinction by DCS

If DCS facilitates extinction by acting as an agonist at the strychnine-insensitive glycine recognition site, then the effect of DCS should be blocked by a strychnine-insensitive glycine site antagonist. To test this, 28 rats were matched into 4 groups of 7 animals each based on the pre-extinction test. 24 hours later, each rat was injected with either saline or HA-966 (6 mg/kg) followed 10 minutes later by a second injection of either saline or DCS (15 mg/kg). This dose was chosen based on pilot experiments suggesting that higher doses of HA-966 alone blocked extinction, thereby complicating interpretations of interactive DCS/HA-966 effects. Thirty minutes later, rats received a single session of extinction training and, 24 hours later, were tested for fear-potentiated startle with no drug injections.

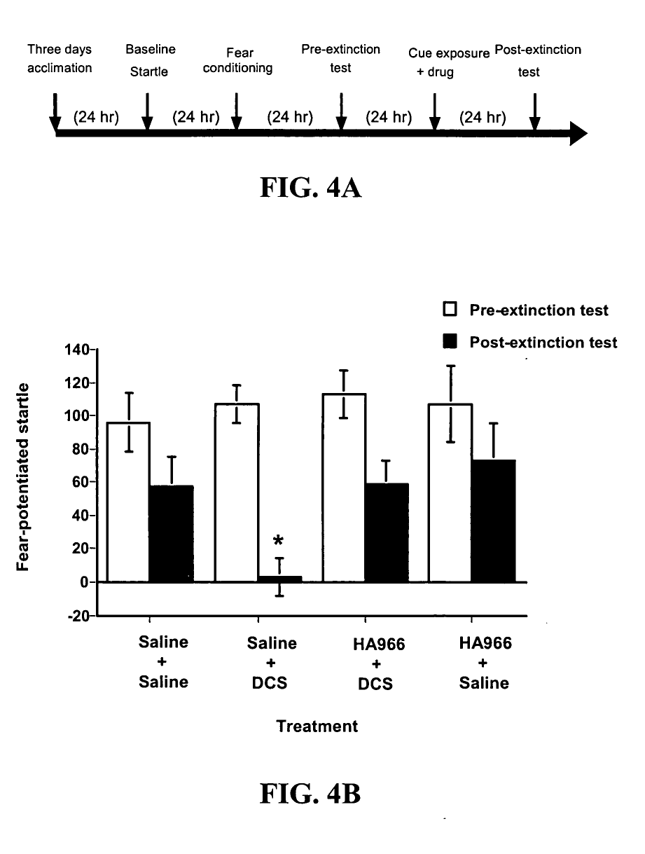

HA-966 completely blocked the enhancement of extinction produced by DCS, but did not itself influence extinction when administered alone (FIG. 4B). Replicating findings from Examples 2 and 3, fear-potentiated startle was significantly lower in rats injected with saline+DCS compared to rats injected with saline+saline (t(12)=2.73). Fear-potentiated startle in rats injected with HA-966+DCS was not significantly different from fear-potentiated startle in rats injected with saline+saline, but was significantly different from fear-potentiated startle in rats injected with saline+DCS (t(12)=3.35). Overall, these results suggest that the facilitatory effect of DCS on extinction is most likely mediated by the NMDA receptor.

### Example 5

### Effect of Pre-Test DCS and HA-966 Administration on Fear-Potentiated Startle

This example evaluated whether the effect of DCS or HA-966 might be secondary to effects on fear itself or on CS processing. For example, if DCS increases CS-elicited fear, this might facilitate extinction by increasing the discrepancy between what the CS predicts and what actually occurs (Wagner and Rescorla (1972) “Inhibition in Pavlovian Conditioning: Application of a Theory,” in Inhibition and Learn., eds. Boakes and Halliday (Academic Press, London)). If HA-966 interferes with visual processing, this might block extinction produced by non-reinforced exposures to the visual CS. To evaluate these possibilities, 17 rats (Saline, N=5; DCS, N=6; HA-966, N=6) were acclimated, tested for baseline startle, and fear-conditioned as previously described. 24 hours later, rats were injected with saline, DCS (15 mg/kg), or HA-966 (6 mg/kg). 30 (for DCS) or 40 (for HA-966) minutes after the injections, rats were tested for fear-potentiated startle.

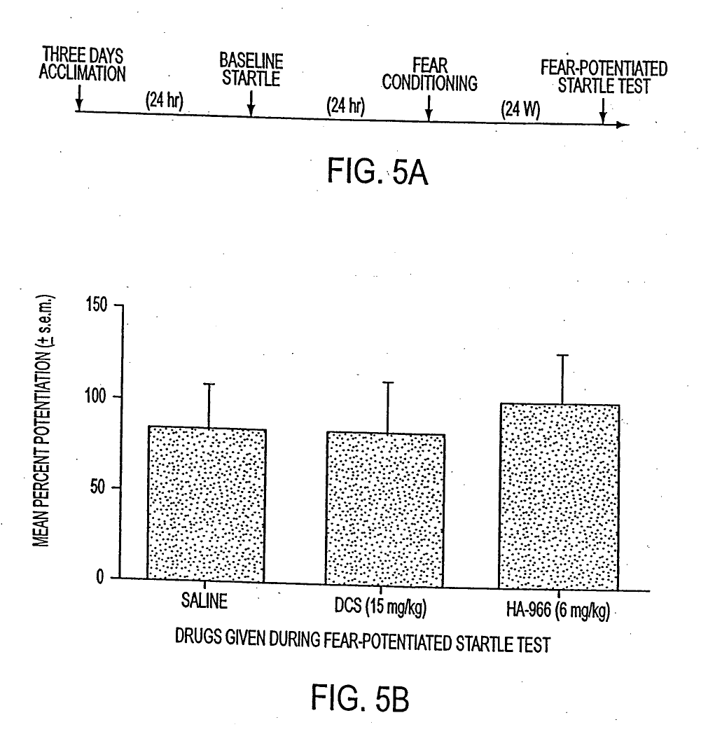

As shown in FIG. 5B, neither DCS nor HA-966 significantly influenced fear-potentiated startle when injected prior to testing. Thus, it is unlikely that these compounds influence extinction by increasing fear or by disrupting CS processing.

### Example 6

### Effect of Intra-Amygdala DCS Infusions on Extinction

Previous studies indicate that NMDA receptors in the amygdala play a critical role in the extinction of conditioned fear (Falls et al. (1992) J. Neuroscience 12:854-863; Lee and Kim (1998) J. Neuroscience 18:8444-8454). It is possible that the effect of systemically administered DCS reported in the above examples was mediated by actions at amygdala NMDA receptors. To determine if the effect of systemically administered DCS would be mimicked by intra-amygdala DCS infusions, 36 rats with intra-amygdala cannulations received fear conditioning, extinction training, and testing for fear-potentiated startle as previously described. Fifteen minutes before being placed into the test chamber for extinction training, rats were infused with either phosphate-buffered saline (PBS) or DCS (10 μg/side) (preliminary findings suggested a weak effect of 1 μg/side and a more potent effect of 10 μg/side). One group of PBS-infused rats and one group of DCS-infused rats received extinction training. An additional group of PBS- and an additional group of DCS-infused rats were not placed in the test chamber and did not receive extinction training. Note that this procedure differed from that of Example 3 in which control rats received context exposure. Because context exposure constitutes context extinction, and because we were particularly concerned in this example that intra-amygdala DCS infusions might be associated with neurotoxicity, we wanted to ensure that any loss of fear-potentiated startle following intra-amygdala infusions could unambiguously be attributed to amygdala damage. If, for example, control rats that had received context extinction showed a reduction of CS-elicited fear, it would be unclear if this was attributable to a DCS-induced lesion or due, instead, to an unintended effect of context extinction on fear to the visual CS. Rats in all groups were tested 24 hours later without drug infusions.

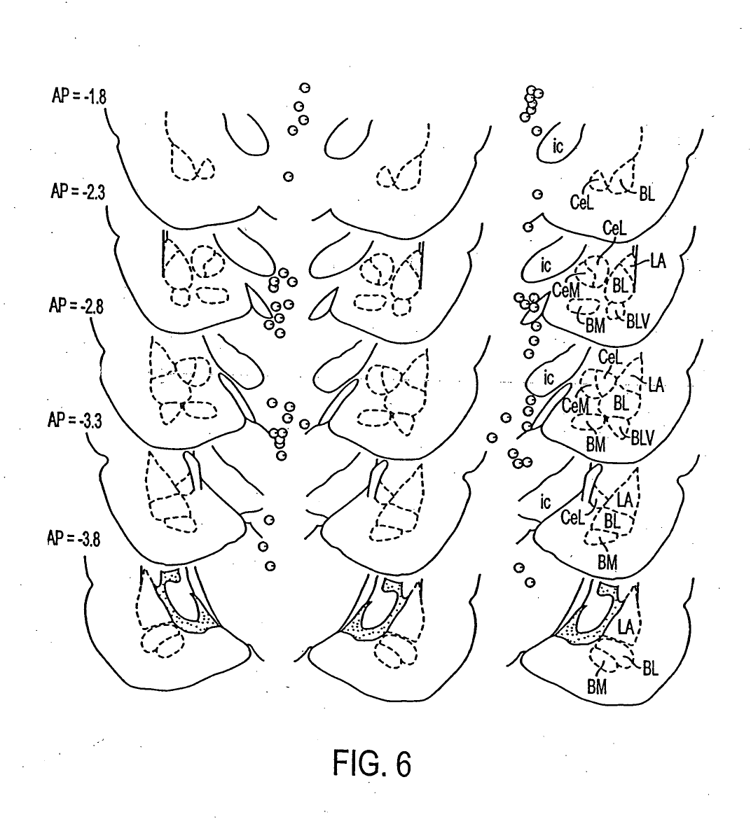

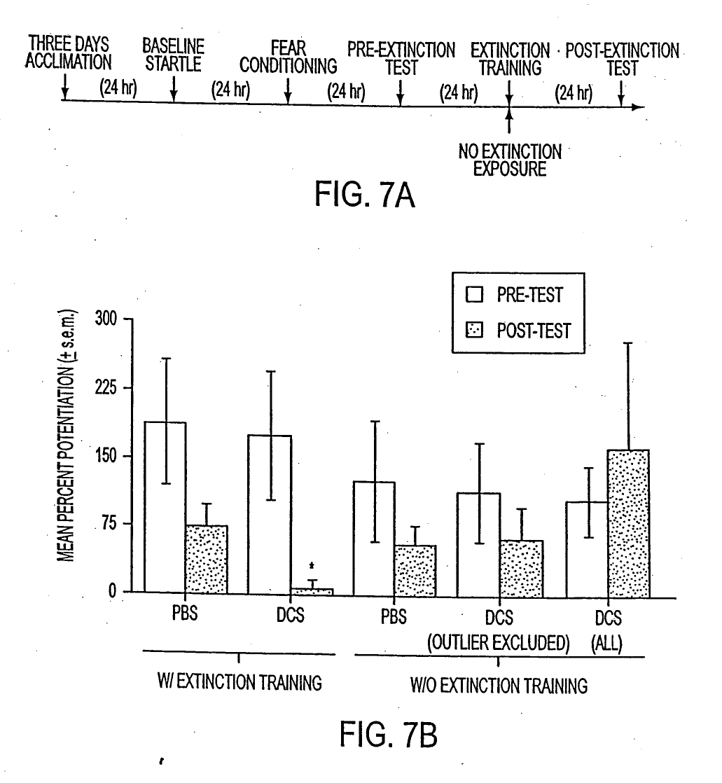

Behavioral data for 10 rats were excluded because the placements for these rats were located outside of the amygdala, resulting in group N's of 9 (PBS—extinction), 9 (DCS—extinction), 4 (PBS—no extinction), and 4 (DCS—no extinction). Placements for the remaining rats are shown in FIG. 6, and the behavioral results are shown in FIG. 7. ANOVA indicated a significant Treatment (DCS versus PBS) X Training (extinction versus no extinction) interaction, F(1, 22)=5.05. Fear-potentiated startle was significantly lower in rats that received intra-amygdala DCS infusions prior to extinction training compared to rats that received intra-amygdala PBS infusions prior to extinction training, t(16)=2.49, and was also significantly lower than in rats that received DCS without extinction training, t(1)=2.36. Fear-potentiated startle was not significantly different in rats that received PBS versus DCS infusions and no extinction training. The latter result suggests that the effect of DCS in rats that received extinction training is not attributable to neurotoxic DCS effects insofar as this would have disrupted fear-potentiated startle in both groups. In fact, fear-potentiated startle was unusually high in non-extinguished rats that received DCS infusions. This was largely attributable to a single rat with a percent increase score of 465%. Even with this outlier excluded, fear-potentiated startle was not significantly different in rats that received PBS versus DCS infusions and no extinction training. As before, however, fear-potentiated startle was significantly lower in rats that received intra-amygdala DCS infusions prior to extinction training compared to rats that received intra-amygdala DCS infusions without extinction training (t(10)=2.34).

Discussion—Examples 1-6

The primary finding of these experiments is that DCS, a partial agonist at the strychnine-insensitive glycine-recognition site on the NMDA receptor complex, facilitates extinction of conditioned fear following either systemic injections (Examples 2, 3, and 4) or intra-amygdala infusions (Example 6). Because DCS reduced fear-potentiated startle only in rats that concurrently received extinction training (Examples 3 and 6), the effects of DCS cannot readily be attributed either to DCS-related neurotoxicity or to anxiolytic drug actions still present 24 hours after drug administration (i.e., during testing). The blockade of DCS's facilitatory influence on extinction by the glycine recognition site antagonist, HA-966, strongly suggests that the effect of DCS was mediated by interactions with the NMDA receptor (Example 4). This seems particularly likely insofar as the dose of HA-966 used did not, on its own, increase fear-potentiated startle. Thus, the ability of HA-966 to reverse DCS effects on extinction cannot be attributed to a summation of independent facilitatory and disruptive effects, mediated by actions on different systems. The failure of either compound to influence fear-potentiated startle when given prior to testing suggests that their effects on extinction reflect direct effects on learning processes rather than on CS-processing or on fear itself.

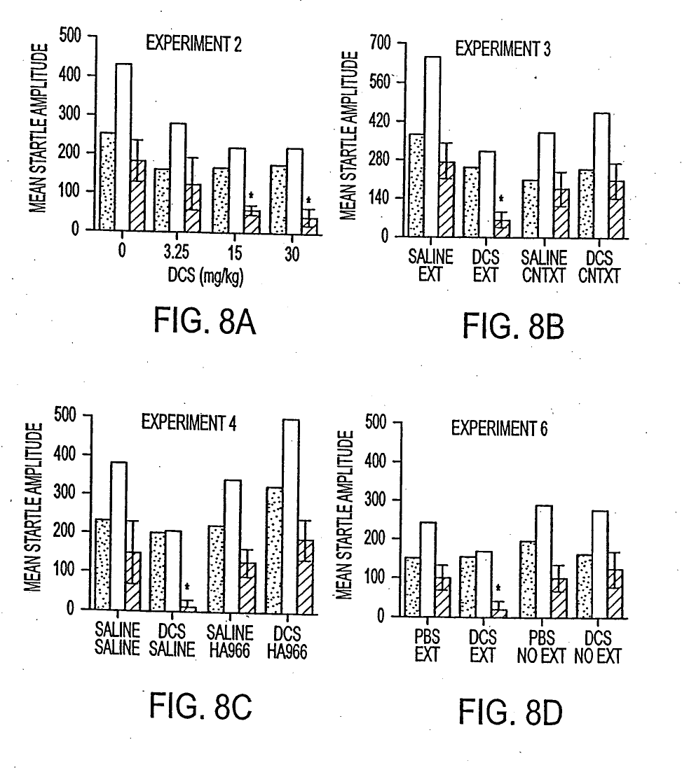

The effects of DCS and HA-966 on extinction are not due to changes in baseline startle. FIG. 8 shows absolute startle values from Examples 2, 3, 4, and 6 (all examples showing drug effects on extinction). Significant drug effects on baseline startle were not found in any experiment when measured in the extinction test 24 hours later. Moreover, the statistical results from analyses of percent potentiation scores were mostly comparable to results obtained using absolute difference scores. Thus, DCS dose-dependently facilitated extinction (F(1,24)=6.03, Example 2). Fear-potentiated startle in the DCS+extinction group was significantly different from fear-potentiated startle in the saline+extinction group in Example 3 (t(12)=3.21), and fear-potentiated startle was comparable in saline and DCS groups that did not receive extinction training. The difference between fear-potentiated startle in DCS+saline-injected versus DCS+HA-966-injected rats approached but did not reach significance (t(12)=1.86, p=0.087, Example 4). Also, fear-potentiated startle was significantly lower in rats that received intra-amygdala DCS infusions prior to extinction training compared to rats that received PBS infusions (t(16)=2.24, Example 6).

Findings implicating amygdala NMDA receptors in both excitatory fear conditioning and conditioned fear extinction are of considerable theoretical interest. Evidence that the extinction of conditioned fear memories might be accelerated by NMDA receptor agonists is also of considerable clinical interest. In clinical populations, a reduced ability to extinguish conditioned fear associations might contribute to the persistence of maladaptive fear and may reduce the effectiveness of therapeutic interventions that rely upon extinction processes (e.g., systematic desensitization, exposure, and imagery therapies). The results reported here suggest that the effectiveness of these traditional clinical approaches might be facilitated by pharmacological interventions that promote extinction.

### Example 7

### Clinical Trial of DCS Augmentation of Behavioral Exposure Therapy for Specific Phobia

Acrophobia, or fear of heights, has been shown to be responsive to virtual reality exposure (VRE) therapy (Rothbaum et al. (1995) Am. J. Psychiatry 152(4):626-628), and VRE therapy has been well validated for different specific phobias and for post-traumatic stress disorder (Rothbaum et al. (1995) Am. J. Psychiatry 152(4):626-628; Rothbaum et al. (2000) J. Consult. Clin. Psych. 68(6):1020-1026). With VRE for fear of heights, it was shown that there were significant improvements on all outcome measures for the treated as compared to the untreated groups (Rothbaum et al. (1995) Am. J. Psychiatry 152(4):626-628). Treated participants in this study reported a positive attitude toward treatment, whereas untreated participants reported negative attitudes. VRE treatment for fear of flying demonstrated that VR treatment was equivalent to standard in vivo exposure therapy, both of which showed significant superiority to waitlist control on all outcome measures (Rothbaum et al. (2000) J. Consult. Clin. Psych. 68(6):1020-1026). In these studies, patients appear to improve steadily across sessions as noted by the decrease in subjective discomfort across sessions as would be expected with incremental habituation or extinction to the fearful stimulus.

In this example, acute treatment with DCS prior to psychotherapy is used to enhance the effects of VRE therapy. Specifically, an acute dose of DCS is given to a patient shortly before each individual therapy session over two weekly sessions to enhance the final level of VRE treatment efficacy. In this example, a 50 mg or 500 mg dose of DCS is given to a patient on an acute basis prior to psychotherapy.

Patient Selection

Although the majority of patients with fear of heights are expected to be simply phobic, it is expected that a substantial minority may be agoraphobic. In this example, a patient must meet DSM-IV criteria for specific phobia, situational type (i.e., fear of heights) or panic disorder with agoraphobia in which heights are the feared stimulus, or agoraphobia without a history of panic disorder, in which heights are the feared stimulus.

Treatment Schedule

A patient is treated once per week for 2 weeks, with a 50 mg or 500 mg DCS dose administered only on the day of therapy, approximately 4 hours before the initiation of therapy. Thus a patient receives only two doses of medication or placebo total over the 2-week period.

Subjects receive virtual reality exposure therapy (VRE) to a series of footbridges over a canyon and a glass elevator that rises 49 floors (Rothbaum et al. (1995) Am. J. Psychiatry 152(4):626-628). During VRE sessions, the patient wears a head-mounted display with stereo earphones that provides visual and audio cues consistent with being on a footbridge over a canyon or inside a glass elevator. During therapy, the therapist makes appropriate comments and encourages continued exposure until anxiety has habituated.

During each VRE session, anxiety is rated by subjective units of discomfort (SUDs) on a 0 to 100 scale in which 0 indicates no anxiety and 100 indicates panic-level anxiety. Psychophysiological responses (pulse, BP, GSR) are monitored throughout each exposure session.

Assessment Methods

A patient's response to a combination therapy session of DCS and VRE may be assessed using any of the methods listed below. Table 1 shows an assessment schedule for a patient done both before and after the combination therapy.

a) Interviews

The Initial Screening Questionnaire (Rothbaum et al. (1995) Am. J. Psychiatry 152(4):626-628) is a short screening instrument that is used to screen initial phone inquiries to identify those likely meeting study criteria for fear of heights.

The Structured Clinical Interview for the DSM-IV (Spitzer et al. (1987) Structured Clinical Interview for DSM III-R(SCID) (New York State Psychiatric Institute, Biometrics Research, New York)) is administered to diagnose and screen for various DSM-III-R axis I disorders (e.g., schizophrenia) as well as establish co-morbid diagnoses.

The Clinical Global Improvement (CGI) Scale is a global measure of change in severity of symptoms. The scale is bipolar with 1=very much improved; 7=very much worse; and 4=no change. It has been used extensively in clinical trials for a variety of psychiatric patients (Guy (1976) ECDEU Assessment Manual for Psychotherapy (revised ed., National Institute of Mental Health, Bethesda, Md.)).

b) Self-report Measures

The Acrophobia Questionnaire (AQ) is a short self-report questionnaire assessing specific symptoms of fear of heights. It is given weekly prior to VRE.

The Attitude Towards Heights Questionnaire (ATHQ) is a separate self-report scale that measures slightly different aspects of avoidance, and other fear of heights related phenomena.

The Rating of Fear Questionnaire (RFQ) (Rothbaum et al. (1995) Am. J. Psychiatry 152(4):626-628) is used to further assess level of fear related to heights in general and the VRE therapy.

The State-Trait Anxiety Inventory (STAI; Spielberger et al. (1970) Manual for the State-Trait Anxiety Inventory (self-evaluation questionnaire) (Consulting Psychologists Press, Palo Alto, Calif.)) is comprised of 40 items divided evenly between state anxiety and trait anxiety. The authors reported reliability for trait anxiety was 0.81; as expected, figures were lower for state anxiety (0.40). Internal consistency ranges between 0.83 and 0.92.

The Beck Depression Inventory (BDI; Beck et al. (1961) Archives of Gen. Psych. 4:561-571) is a 21-item self-report questionnaire assessing numerous symptoms of depression. The authors report excellent split-half reliability (0.93), and correlations with clinician ratings of depression range between 0.62 and 0.66.

c) Therapist Measure

The subjective units of discomfort (SUDs) is scored by the therapist based on the participant's report during the VRE at 5 minute intervals. SUDS are rated on a 0 to 100 scale in which 0 indicates no anxiety and 100 indicates panic-level anxiety

The Behavioral Avoidance Test (BAT) consists of a brief re-exposure to heights via the Virtual Reality environment, in which the therapist assesses the patients subjective level of fear and avoidance of heights.

d) Psychophysiological Measures

Measurement of heart rate (HR) is performed and stored by a non-invasive, computer controlled monitoring device for assessment of autonomic reactivity during VRE.

Measurement of blood pressure (BP) is performed by a non-invasive, computer controlled sphygmomanometer for assessment of vascular tone and autonomic reactivity during VRE

Measurement of galvanic skin conductance (GSR) is performed by a non-invasive, computer controlled monitoring device for assessment of autonomic fear responsivity during VRE.

TABLE 1

| Assessment Session | Measures |
|---|---|
| Prior to entry | Consent form |
| | SCID |
| Pre-treatment Assessment | Acrophobia Questionnaire |
| | Attitude Towards Heights Questionnaire |
| | Ratings of Fear Questionnaire |
| | Behavioral Avoidance Test |
| | BDI |
| | STAI |
| Weekly VRE Therapy Sessions | Psychophysiologic measures (HR, BP, |
| (x2) | GSR) |
| | SUDs |
| Post-VRE Assessments and 6 | Acrophobia Questionnaire |
| Month Follow up Assessment | Attitude Towards Heights Questionnaire |
| | Ratings of Fear Questionnaire |
| | CGI |
| | Behavioral Avoidance Test |

### Example 8

### Clinical Trial of DCS Auqmentation of Behavioral Exposure Therapy for Specific Phobia

The present example provides results from a clinical study based on the protocol described in Example 7.

Patient Selection and Group Assignment

Twenty-eight volunteer patients (or participants) were recruited from the general community with no currently active psychiatric disorders except for acrophobia. The diagnosis of acrophobia (a subtype of Specific Phobia), requires an excessive or unreasonable fear of heights that interferes significantly with the person's normal routine and functioning, and is characterized by severe anxiety in the presence of height situations.

One participant did not return after the pre-assessment. Therefore, 27 patients (11 male, 16 female) were randomly assigned to three treatment groups via a predetermined and blinded order of treatment assignment. These groups were: 1) Placebo+VRE Therapy (n=10); 2) 50 mg DCS+VRE Therapy (n=8); and 3) 500 mg DCS+VRE Therapy (n=9). Treatment condition was double-blinded, such that the subjects, therapists, and assessors were not aware of assigned study medication condition. The blind was maintained throughout the duration of the study. All 27 patients completed pretreatment, both therapy sessions, and the 3-month follow-up assessment. Pre-treatment data are listed in Table 2.

TABLE 2

| Characteristic | Placebo (N = 10) | DCS (N = 17) | p value |
|---|---|---|---|
| Age | 44.8 ± 2.3 | 46.4 ± 2.8 | 0.68 |
| DSM-IV Dx | 2.1 ± .69 | 1.6 ± .24 | 0.41 |
| GAF | 64.7 ± 1.3 | 65.1 ± .72 | 0.76 |
| BDI | 7.7 ± 4.4 | 4.2 ± 1.1 | 0.34 |
| STAI-state | 34.2 ± 5.6 | 33.9 ± 2.7 | 0.96 |
| STAI-trait | 31.7 ± 4.5 | 31.4 ± 1.9 | 0.95 |
| AAQ | 65.8 ± 6.2 | 73.4 ± 5.6 | 0.39 |
| AAVQ | 18.7 ± 2.7 | 24.2 ± 2.5 | 0.17 |
| ATH | 54.4 ± 1.7 | 53.9 ± 1.7 | 0.84 |

Abbreviations:
DSM-IV DX: number of DSM-IV diagnoses by SCID;
GAF: Global Assessment of Functioning Scale;
BDI: Beck Depression Inventory;
STAI: state-trait anxiety scale;
AAQ: acrophobia anxiety questionnaire;
AAVQ: acrophobia avoidance questionnaire;
ATH: attitude towards heights inventory.
Values are given as mean ± sem.

Patient Assessment

Patients were examined with a battery of screening tests (Ressler et al., (2004) Archives of Gen. Psych. 61:1136-1144). Acrophobia and other psychiatric diagnoses were determined by interview with the Structured Clinical Interview for DSM-III-R. The Acrophobia Questionnaire with Avoidance (AAVQ) and Anxiety (AAQ) subscales were used to examine their fear of heights (Cohen D. (1977) Behav. Therapy 8:17-23). The Attitudes Towards Heights Inventory (ATHI) was used to examine their fear of heights. The Beck Depression Inventory (BDI) and the State/Trait Anxiety Inventory (STAI) were used to examine their general levels of depression and anxiety. Overall global improvement was assessed with the Clinical Global Improvement (I) Scale (CGI). During the initial screen, patients also had limited but structured exposure to the virtual reality height environment during a Behavioral Avoidance Test (BAT), in which they reported on a 0-100 scale (100 being the most intense fear) their subjective units of discomfort (SUDS) for each floor (floors 1-19) of the virtual glass elevator.

Electrodermal skin conductance fluctuations (SCF) were measured as described (Ressler et al., (2004) Archives of Gen. Psych. 61:1136-1144). Skin conductance correlates with perioperative stress. Finger electrodes (ProComp Module, Thought Technology Ltd., Montreal) were worn by the subject during the initial and post-treatment behavioral assessment tests. Data are reported as number of SCFs per minute of exposure. SCF was averaged over the entire exposure and presented as fluctuations per minute. Each fluctuation was defined as ≧2 second deviation of 0.05 μS from the local mean (average baseline+/−30 seconds). Follow-up analyses also examined fluctuations as defined by a ≧2 second deviation of 5% greater or less than the local mean.

Medication

DCS (Seromycin, 250 mg, Eli Lilly Pharmaceuticals) was reformulated into 50 mg or 500 mg capsules which were identical in appearance to placebo capsules.

No adverse events occurred during the study. Although reports of side effects were not systematically obtained, subjects were routinely asked if they were experiencing any difficulties. Upon breaking the blind no difference was found between subjects reporting side effects with placebo or DCS.

Treatment Schedule

With VRE for fear of heights, a virtual glass elevator was used in which patients stood while wearing a VRE helmet and were able to peer over a virtual railing. Computerized effects give a real sense of increase in height as the elevator rises. Previous work had demonstrated improvements on all acrophobia outcome measures for treated subjects (as compared to untreated groups) after seven weekly, 35-45 minute therapy sessions (Rothbaum et al. (1995) Am J Psychiatry 152:626-8).

Patients underwent two 35-45 minute therapy sessions, which is a suboptimal amount of exposure therapy for acrophobia. These two therapy sessions were separated by 1-2 weeks (average=12.9 days). Patients were instructed to take a single pill of study medication (placebo, 50 mg DCS, or 500 mg DCS) two to fours hours before each therapy session, such that only two pills were taken for the entire study. There were no adverse events reported from either group taking placebo or drug prior to exposure therapy.

A mid-treatment assessment occurred one week after the first treatment (average=7.2 days). A post-treatment assessment was performed 1-2 weeks after the final therapy session (average=11.5 days), and an additional follow-up assessment was performed three months after the therapy (average=107.5 days).

Assessment

Patients, therapists and assessors were kept blind to treatment condition throughout the study. All data were entered into the SPSS statistics package by research assistants also blind to condition. Pre-treatment variables (Table 2) were analyzed using t-tests for independent samples. Post-treatment variables (skin conductance fluctuations, AAQ, AAVQ, ATH, CGI, and number of self exposure to heights) were analyzed using one-way ANOVA or Repeated Measures ANOVA with time and drug condition as separate factors.

Specific comparisons of different floors and SUDS within treatment sessions were performed with one-way ANOVA with the between-subjects factor of drug vs. placebo group. The effect of interaction between drug group and different floors or drug group and different time points on the SUDS score (FIG. 1) was performed using multivariate analysis with repeated measures with floor or time as the repeated within-subjects factor and drug condition the independent between-subjects factor. The effect of these interactions on SUDS as the outcome variable for the pre-post analysis (FIG. 2) was performed with an overall ANOVA with pre-post difference and floor as within-subjects factor and drug group as between-subjects factor.

Results for Example 8

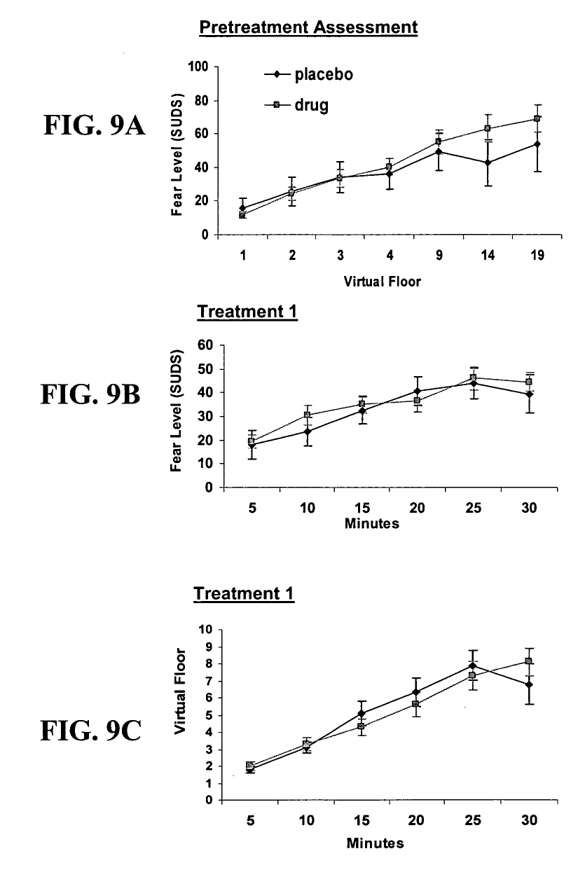

Twenty-seven patients completed the two therapy sessions, with ten subjects randomly assigned to placebo (5 males, 5 females), and seventeen subjects randomly assigned to DCS (6 males, 11 females). At the pretest assessment there was no difference in age, number of DSM-IV diagnoses, global assessment of functioning (GAF), or scores on the BDI, STAI-state, or STAI-trait between placebo and drug groups (Table 2). There was also no difference in initial acrophobia measures (Table 2) or in SUDS levels at different floors within the virtual elevator environment (FIG. 9A).

Following treatment, statistically significant differences were found between placebo and drug groups for almost every primary outcome measure. In the results below, statistics are presented for ANOVA measures with the drug groups both separated and combined. Analysis of the data indicated that there were no significant differences between the 50 mg and 500 mg drug groups for the primary outcome measures of acrophobia (ANOVA, p values>0.5); therefore, the data in the figures are presented with drug groups combined.

Because no direct anxiolytic effect of DCS was anticipated based on preclinical studies and also because there was no retention interval to allow facilitative effects of DCS on extinction learning, no effects of DCS were anticipated for session one. Consistent with this, no differences were found between groups in SUDS level during the first therapy session (FIG. 9B). During the therapy sessions, patients have some control over how high the elevator is allowed to rise, permitting an analysis of avoidance of heights. During this first session, no differences were found in the highest floor attained at different time points (FIG. 9C). These findings indicate that the presence of DCS during the therapy session did not affect level of fear or avoidance of fear during the therapy.

During the second session, patients in the DCS group experienced lower SUDS than the placebo group (SUDS at 5 minutes, F(1,25)=7.1, p≦0.01), and they elevated to higher floors after 20 minutes (mean floor for placebo=13.0, for DCS=15.9, F(1,25)=6.3; p≦0.01). This suggests that during the second session, there was less fear and avoidance in the group that had received DCS during the first session. The DCS group also showed more improvement as measured by participant scores on the CGI scale at the second session (placebo=2.8 vs DCS=2.25; F(1,25)=5.2; p≦0.05). Accordingly, the results show that DCS enhances extinction of fear within the virtual environment.

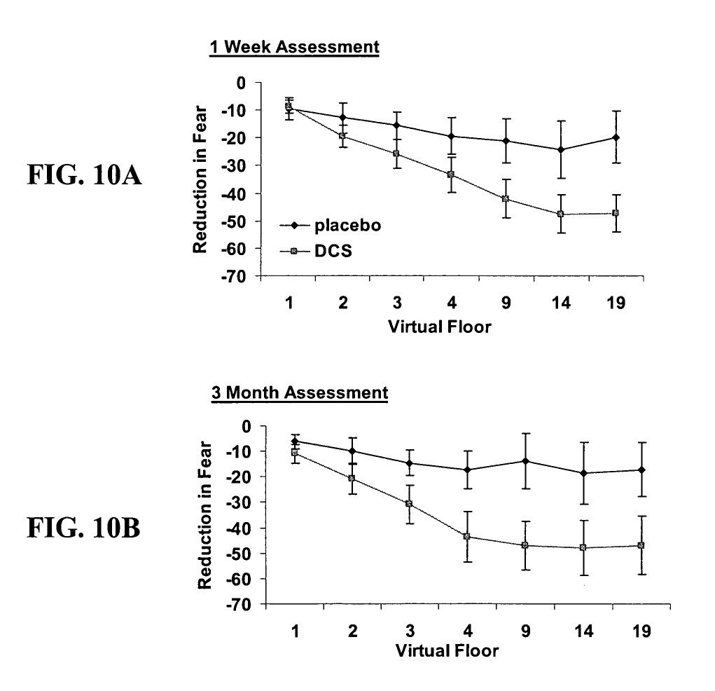

One week after the second session a post-treatment assessment was performed in the absence of drug and the difference scores between the post-treatment and pre-treatment were examined. The group that received DCS during the therapy sessions showed significantly less fear of heights as determined by SUDS at successive elevator floors during the BAT VR assessments (FIG. 10A; F(6,150)=3.8, p≦0.001). This difference was also seen if the two separate doses of drug were analyzed separately with a repeated measures ANOVA (F(12, 144)=2.7, p≦0.01). The continued decrease in fear within the virtual environment in the absence of DCS demonstrates that the enhancement of extinction in humans with DCS is not state-dependent. These data suggest that two sessions of VRE therapy in combination with DCS for fear of heights is sufficient for extinction of fear within the virtual environment (FIG. 10A).

To evaluate how DCS would affect retention of extinction, as well as whether it would generalize to real life situations outside the VR environment over time, subjects were asked to return for a follow-up session 3 months after their VRE treatment. Twenty-one of the 27 completing patients returned for follow-up assessment [8 placebo (80% of enrolled), 13 DCS (77% of enrolled)]. Analysis of the pre-treatment data and the one-week post-treatment assessments showed that there were no significant pre- or post-treatment differences on anxiety or fear measures between those that returned for follow-up and the six that did not.

At the follow-up assessment, subjects were tested again in the absence of DCS for their level of fear in the virtual elevator environment with the BAT. It was found that patients who received DCS maintained the specific decrease in fear to the virtual environment over the 3-month period as determined by SUDS during the exposure to virtual heights (FIG. 2B; F(6,102)=2.4, p≦0.05). No significant differences between the two different drug doses were found. This suggests that the extinction of fear that was enhanced in the drug group during the two therapy sessions was relatively robust and lasting.

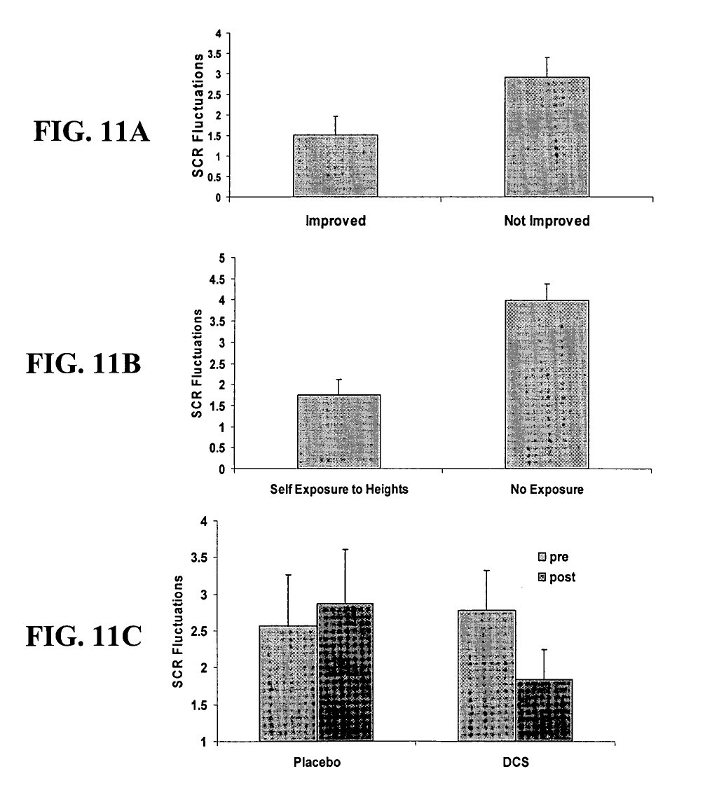

The number of spontaneous fluctuations of skin conductance is a common measure of emotional arousal and anxiety, such that those with more fear or anxiety typically show more spontaneous reactivity or fluctuation in their baseline skin conductance during provocation. Consistent with this pattern, during the post-treatment behavioral assessment tests, it was found that the number of spontaneous fluctuations correlated with the measures of subjective improvement in fear of heights. Those reporting “much” or “very much” improvement at the initial post-treatment assessment test showed significantly fewer spontaneous fluctuations than did those who reported no improvement or worsening (FIG. 1A; F(1,19)=4.5, p≦0.05; linear regression, r=0.44). Additionally, those who showed less avoidance of heights in the real world since treatment, as indicated by increased likelihood of exposing themselves to real-world heights, also showed fewer spontaneous fluctuations than did those who did not self-expose since treatment (FIG. 11B; F(1,19)=8.26; p≦0.01; linear regression, r=0.55).

It was also found that those subjects given DCS during exposure therapy had a significant decrease in average spontaneous fluctuations from pre- to post-treatment (FIG. 11C; paired t-test, p≦0.05) compared to those given placebo during the treatment (p≧0.5). Subsequent analysis of skin conductance fluctuations using the criterion of a 5% change from baseline in skin conductance instead of an absolute 0.05 μS difference also demonstrated a significant time X treatment effect (repeated measure ANOVA, F(1, 19)=8.0, p≦0.01). These data suggest that the improvement in extinction of fear achieved with DCS augmentation during exposure was evident in both subjective and objective physiological measures of fear.

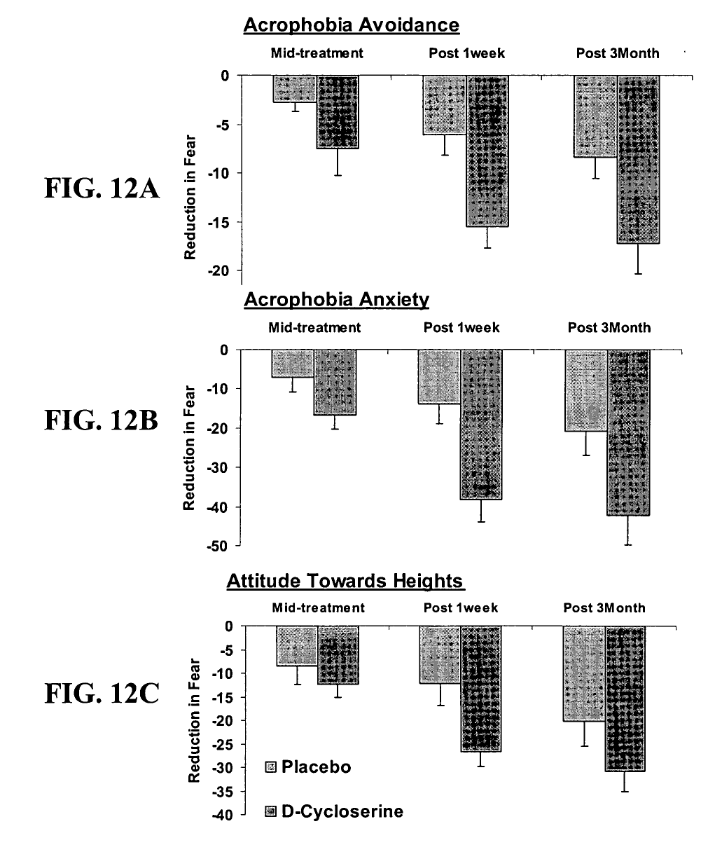

To examine the ability of the virtual reality exposure to heights to reduce symptoms of acrophobia in the real world, standard outcome measures of acrophobia that are not specific to the virtual environment were utilized. These measures were taken at the pretreatment assessment, the mid-treatment assessment between the two therapy sessions, one to two weeks post-treatment, and three months post-treatment. These measures were always taken in the absence of medication, and the questionnaires referred to subjects' symptoms of acrophobia in the real world, not the virtual environment. FIG. 12 shows the reduction of fear as measured by difference scores between each post-treatment measure and the pretreatment baseline measure for placebo and DCS groups.

For all principle outcome measures, significant improvements in the DCS groups as compared to placebo group were found in this repeated measure analysis. This is true for generalized avoidance of heights measures (AAVQ; F(1, 19)=6.1, p≦0.02), anxiety due to heights (AAQ; F(1, 19)=7.9, p≦0.01), and general attitudes towards heights (ATHI; F(1, 19)=4.9, p≦0.04). These significant primary outcomes were also seen when the placebo, 50 mg DCS, and 500 mg DCS subject groups were separated (AAVQ: F(2, 18)=5.9, p≦0.01; AAQ: F(2, 18)=4.0, p≦0.04; ATHI: F(2, 18)=2.5, p≦0.1). These data suggest that the enhanced extinction that occurred during the initial two therapy sessions was robust and lasting, and also that it was capable of generalization to real-world height situations during the three months that followed the therapy.

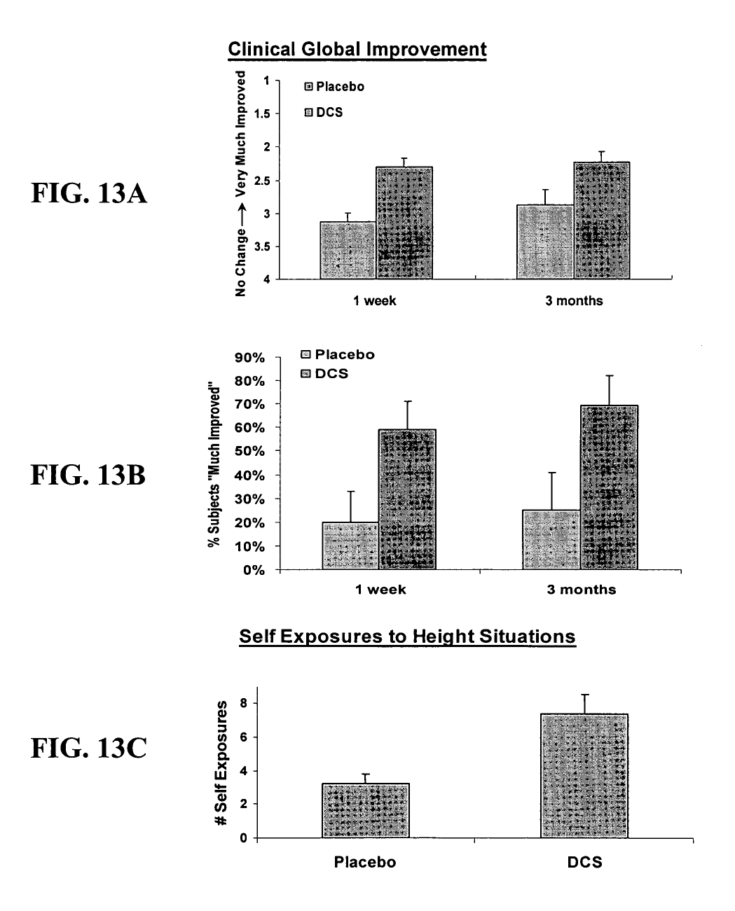

The final analyses examined general measures of overall improvement in acrophobia as well as evidence for functional gains in the subjects' lives at the 3-month follow-up assessment (FIG. 13). Average scores on the CGI scale were significantly higher at the one-week and three-month follow-up sessions as analyzed with a repeated measures ANOVA (FIG. 5A; DCS vs placebo F(1, 19)=11.6, p≦0.005). Analysis of placebo, 50 mg DCS, and 500 mg DCS subject groups separately also revealed significant differences (F(2, 18)=5.6, p≦0.01). Furthermore, as seen in FIG. 13B, the DCS group showed significantly greater percentages of subjects reporting ‘much improvement’ or ‘very much improvement’ compared to the placebo group at one week and at three months (FIG. 13B; repeated measures ANOVA: overall drug effect F(1, 19)=11.5, p≦0.005, but no drug×time interaction).

A critical measure of functional improvement is the actual number of times the subjects exposed themselves to previously fear-inducing heights in the period following the treatment. Previous studies have demonstrated that subjects successfully treated for acrophobia will expose themselves to heights in the real world following treatment much more than will those who are still fearful of heights. When subjects were asked to report the number of significant exposures (e.g. peering over a high railing, bridge, etc.) that they had experienced since the completion of treatment, subjects receiving DCS during treatment reported over twice as many exposures than did those receiving placebo (FIG. 13C; DCS vs placebo, F(1, 18)=7.7, p≦0.01, with values of F(2, 18)=3.6, p≦0.05 when the DCS-treated groups were analyzed separately).

Discussion for Example 8

These data demonstrate that DCS facilitates the effects of exposure therapy for the treatment of acrophobia. Patients in the DCS group showed some evidence of enhanced extinction after only a single dose of medication and therapy. Following two doses of medication and therapy, they showed significant reductions in levels of fear to the specific exposure environment in both subjective and objective physiological measures of fear. Finally, three months following the two treatment sessions, the DCS patients showed significant improvements on all general acrophobia measures, their own self-exposures in the real world, and their impression of clinical self-improvement.

The data indicate that patients receiving DCS experienced no significant change in anxiety or fear during the exposure paradigm so that the enhancement of extinction is not due simply to altered intensity of exposure. Additionally, the placebo and drug groups were evenly matched on all measures prior to the study (Table 2) suggesting that pre-treatment variables did not contribute to the differential improvement in groups. The slightly higher, but non-significant, depression scores in the placebo group compared with the DCS group (BDI=7.7 vs 4.2) raised the issue of whether subclinical depression could account for some of the differences seen. To test this hypothesis, all the primary outcomes were re-analyzed with pre-treatment BDI as a co-variate. In all cases (one- or three-week SUDS, SCF, AAQ, AAVQ, ATH, CGI, and self-exposure), none of the covariate analyses were significant (p's=0.12-0.88). Therefore the data presented here specifically support the role of DCS during exposure therapy contributing to the resultant enhanced improvement in acrophobia.

It is interesting to note that no apparent increase in extinction was observed during the treatment session, but only between sessions. It has been suggested that the NMDA-dependent phase of extinction training occurs during the post-extinction consolidation period (Santini et al. (2001) J. Neurosci. 21:9009-17).

Although specific phobia provides an easily testable disorder that is amenable to behavioral exposure therapy, this form of therapy is also the mainstay of treatment for other anxiety disorders such as panic disorder, obsessive compulsive disorder and post-traumatic stress disorder. In addition, the process of extinction of conditioned cues can be important for recovery from disorders of substance dependence.

### Example 9

A subject afflicted with recurring chronic pain was treated for seven days with 10 mg per day amitriptyline hydrochloride, with little effect on the pain. After seven days, the dose of amitriptyline was doubled to 20 mg. After two days on the higher dose, the subject's pain substantially subsided. The subject was subsequently treated with a single dose of a capsule containing 20 mg amitriptyline and 25 mg DCS, in addition to pharmaceutically inactive ingredients. Subsequently, the subject reverted to taking 20 mg amitriptyline (with no DCS) until the amitriptyline treatment had been ongoing for a period of four weeks, before tapering the dose down and then stopping treatment.

### Example 10

A powdered blend of 10 mg zolpidem tartrate and 50 mg DCS, in addition to the following inactive ingredients: hydroxypropyl methylcellulose, lactose, magnesium stearate, microcrystalline cellulose, polyethylene glycol, sodium starch glycolate, and titanium dioxide, was mixed and loaded into a capsule. The capsule was administered to a subject afflicted with recurring transient insomnia. After taking the capsule, the subject experienced a short sleep onset, as well as good sleep quality. For the next five nights, the subject was administered a capsule containing 5 mg zolpidem tartrate, without co-administration of DCS. On the seventh night, the subject was administered a single capsule containing 5 mg zolpidem tartrate and 50 mg DCS, in addition to the following inactive ingredients: hydroxypropyl methylcellulose, lactose, magnesium stearate, microcrystalline cellulose, polyethylene glycol, sodium starch glycolate, and titanium dioxide. The subject discontinued further treatment. The subject's anxiety regarding sleep, particularly after awakening in the middle of the night, was reduced, and the subject found it easier to revert to a sleeping state.

### Example 11

A powdered blend of sildenafil citrate (50 mg) and DCS (50 mg), as well as inactive ingredients including: microcrystalline cellulose, dibasic calcium phosphate, croscarmellose sodium, magnesium stearate, hydroxypropyl methylcellulose, titanium dioxide, lactose, and triacetin, was mixed and loaded into a capsule.

### Example 12

A healthy volunteer subject was administered a capsule containing about 50 mg sildenafil citrate and 50 mg DCS, in addition to inactive ingredients. The pharmaceutical composition was assessed for adverse effects or reactions in the subject, of which none were reported. The pharmaceutical composition was also assessed for its effects on sexual performance enhancement.

Although the foregoing invention has been described in some detail by way of illustration and example for purposes of clarity of understanding, it will be obvious that certain changes and modifications may be practiced within the scope of the representative embodiments of these concepts presented below.

We claim:

1. A method for facilitating extinction of a deleterious, high-anxiety response in a subject, comprising:
(A) administering to said subject a sub-antimicrobial dose of D-cycloserine; and
(B) exposing said subject to an extinction learning event within eight hours of said administering of said sub-antimicrobial dose of D-cycloserine.

2. The method of claim 1, wherein said extinction learning event occurs within eight hours after said administering of said sub-antimicrobial dose of D-cycloserine.

3. The method of claim 1, wherein said extinction learning event occurs within about four hours after said administering of said sub-antimicrobial dose of D-cycloserine.

4. The method of claim 1, wherein said sub-antimicrobial dose of D-cycloserine is administered within about four hours after said extinction learning event.

5. The method of claim 1, wherein said sub-antimicrobial dose of D-cycloserine is administered on an acute basis.

6. The method of claim 1, wherein said subject is a dog.

7. The method of claim 1, wherein said subject is a human.

8. The method of claim 1, wherein said extinction learning event results from extinction training comprising psychotherapy.

9. The method of claim 8, wherein said psychotherapy is selected from the group consisting of exposure-based psychotherapy, cognitive behavioral therapy, and psychodynamically oriented psychotherapy.

10. The method of claim 8, wherein said deleterious, high-anxiety response exacerbates symptoms of a medical disorder selected from the group consisting of anxiety disorders, insomnia, erectile dysfunction, and female sexual dysfunction.

11. The method of claim 10, wherein said medical disorder is an anxiety disorder.

12. The method of claim 10, wherein said anxiety disorder is a phobic disorder.

13. The method of claim 10, wherein said anxiety disorder is post-traumatic stress disorder.

14. The method of claim 10, wherein said anxiety disorder is an addictive disorder.

15. The method of claim 8, wherein said deleterious, high-anxiety response exacerbates symptoms of a medical disorder selected from the group consisting of chronic pain and chronic fatigue syndrome.

16. The method of claim 1, wherein said extinction learning event is not achieved through psychotherapy.

17. The method of claim 1, wherein said extinction learning event comprises a successful sexual outcome.

18. The method of claim 1, wherein said extinction learning event comprises biofeedback therapy.

19. The method of claim 1, wherein said extinction learning event extinguishes a deleterious, high-anxiety response that exacerbates a medical disorder selected from the group consisting of anxiety disorders, erectile dysfunction, female sexual dysfunction, and insomnia.

20. The method of claim 1, wherein said sub-antimicrobial dose of D-cycloserine is between about 0.5 mg/kg and 2.0 mg/kg body weight of subject.

21. The method of claim 20, wherein said sub-antimicrobial dose of D-cycloserine comprises a tablet containing between about 50 mg and 75 mg D-cycloserine.

22. The method of claim 1, wherein said sub-antimicrobial dose of D-cycloserine is administered in a controlled-release formulation.

23. The method of claim 1, wherein a B-complex vitamin is administered in conjunction with said sub-antimicrobial dose of D-cycloserine.

24. The method of claim 1, wherein modafinil is co-administered with said sub-antimicrobial dose of D-cycloserine.
